(19) United States
(12) Patent Application Publication
(10) Pub. No.: US 2008/0287452 A1
(43) Pub. Date: Nov. 20, 2008

(54) HETEROARYL/ARYL PYRIMIDINE ANALOGS AND THEIR USE AS AGONISTS OF THE WNT-BETA-CATENIN CELLULAR MESSAGING SYSTEM

(75) Inventors: Matthew Gregory BURSAVICH, Salt Lake City, UT (US); Adam Matthew GILBERT, Congers, NY (US); Diane Barbara HAUZE, Wayne, PA (US); Charles William MANN, Plymouth Meeting, PA (US); Jeffrey Claude PELLETIER, Lafayette Hill, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/122,268

(22) Filed: May 16, 2008

Related U.S. Application Data
(60) Provisional application No. 60/924,477, filed on May 16, 2007.

(51) Int. Cl. A61K 31/506; C07D 401/14; C07D 403/02

(52) U.S. Cl. 514/252.02; 544/331; 514/275; 514/255.05; 544/238

(57) ABSTRACT

The present invention relates to heteroaryl/aryl pyrimidine analogs, methods of making aryl/heteroaryl pyrimidine analogs, compositions comprising a aryl/heteroaryl pyrimidine analog, and methods for treating canonical Wnt-β-catenin cellular messaging system-related disorders comprising administering to a subject in need thereof an effective amount of a heteroaryl/aryl pyrimidine analog.

### CROSS-REFERENCE TO RELATED APPLICATIONS

This application claims priority benefit of U.S. provisional patent application No. 60/924,477, filed May 16, 2007, the entire disclosure of which is hereby incorporated by reference.

### FIELD OF THE INVENTION

The invention relates to heteroaryl/aryl pyrimidine analogs, compositions comprising a heteroaryl/aryl pyrimidine analog, and methods for treating or preventing disease involving the canonical Wnt-β-catenin cellular messaging system comprising the administration of an effective amount of a heteroaryl/aryl pyrimidine analog.

### BACKGROUND OF THE INVENTION

The Wnt-β-catenin cellular messaging system is essential in many biological processes. It regulates the fate of as-yet undeveloped cells in embryo form. The signals in the messaging system also direct the development of stem cells in adult organisms (e.g. skin cell, bone cell, liver cell, etc.). At the cellular level, the canonical Wnt-β-catenin cellular messaging system regulates morphology, proliferation, motility and cell fate. The Wnt-β-catenin messaging system has a central role in tumorigenesis and inappropriate activation of this system is observed in several human cancers.

Wnt-β-catenin was first described in humans as a protein, which interacts with the cytoplasmic domain of E-cadherin and with α-catenin, anchoring the cadherin complex to the actin cytoskeleton. Then, an additional role for mammalian β-catenin was discovered; namely, as the key mediator of Wnt-β-catenin messaging.

Chronic activation of the Wnt-β-catenin cellular messaging system has been implicated in the development of a variety of human malignancies, including colorectal carcinomas, hepatocellular carcinomas (HCCs), melanomas, and uterine and ovarian carcinomas.

The Wnt-β-catenin cellular messaging system has also been shown to play a role in degenerative diseases such as Alzheimer's disease and bone disorders.

Alzheimer's disease (AD) is the most common age-related neurodegenerative disorder. A massive accumulation of beta-amyloid (Abeta) peptide aggregates has been proposed as pivotal event in AD. Abeta-induced toxicity is accompanied by a variegated combination of events including oxidative stress. The Wnt-β-catenin pathway has multiple actions in the cascade of events triggered by Abeta, and drugs with Wnt-β-catenin activity can be therapeutics for AD treatment.

Various bone disorders are also associated with the Wnt-β-catenin messaging system. Signaling through the Wnt-β-catenin pathway that increases bone mass through a number of mechanisms including renewal of stem cells, stimulation of preosteoblast replication, induction of osteoblastogenesis, and inhibition of osteoblast and osteocyte apoptosis.

As discussed above, agonists of the Wnt-β-catenin messaging system are expected to be novel types of medicaments useful against cell proliferation disorders, bone disorders, and Alzheimer's disease. Thus, it would be advantageous to have new agonists of the Wnt-β-catenin messaging system as potential treatment regimens for Wnt-β-catenin messaging system-related diseases. The instant invention is directed to these and other important ends.

### SUMMARY OF THE INVENTION

In one aspect, the invention provides compounds of the Formula 1:

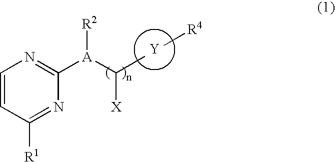

and pharmaceutically acceptable salts, hydrates, and solvates thereof,
wherein

R¹ is aryl, or heteroaryl, both optionally substituted with 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
Y is imidazolyl, triazolyl, pyrazolyl, pyrrolyl, pyridinyl, pyrimidinyl, pyridizinyl, pyrazinyl, phenyl, indolyl, pyrolepyridinyl, all optionally substituted by 1-6 independently selected R⁴ groups; and
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

In another aspect, the invention provides compounds of Formula 1A:

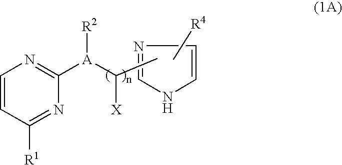

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the imidazolyl ring is optionally substituted with 1-3 independently selected R⁴ groups.

In another aspect, the invention provides compounds of Formula 1B:

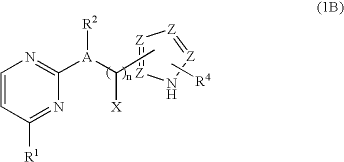

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein each Z is independently N or CH, whereby two of the Z groups are N, thereby forming a triazolyl ring;
   wherein the triazolyl ring is optionally substituted with 1-2 independently selected R⁴ groups.

In another aspect, the invention provides compounds of Formula 1C:

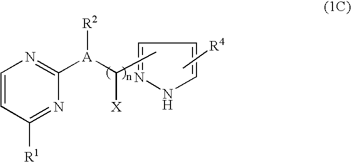

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₃-C₆ alkoxy, CO₂C₁-C₆ alkyl, CN, OH, cycloalkyl, CONH₂, aryl, heteroaryl, COaryl, or trifluoroacetyl;
   wherein the pyrazolyl ring is optionally substituted with 1-3 independently selected R⁴ groups.

In another aspect, the invention provides compounds of Formula 1D:

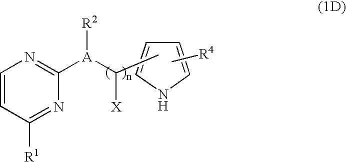

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the pyrrolyl ring is optionally substituted with 1-4 independently selected R⁴ groups.

In another aspect, the invention provides compounds of Formula 1E:

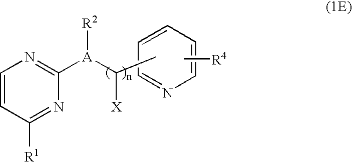

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;

n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;

each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the pyridinyl ring is optionally substituted with 1-4 independently selected R⁴ groups.

In another aspect, the invention provides compounds of Formula 1F:

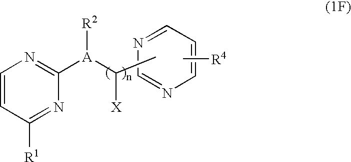

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the pyrimidinyl ring is optionally substituted with 1-3 independently selected R⁴ groups.

In another aspect, the invention provides compounds of Formula 1G:

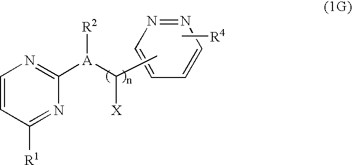

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H s;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the pyridizinyl ring is optionally substituted with 1-3 independently selected R⁴ groups.

In another aspect, the invention provides compounds of Formula 1H:

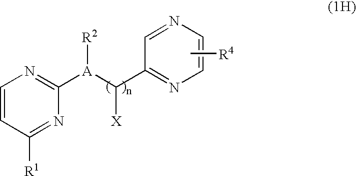

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the pyrazinyl ring is optionally substituted with 1-3 independently selected R⁴ groups.

In another aspect, the invention provides compounds of Formula 1I:

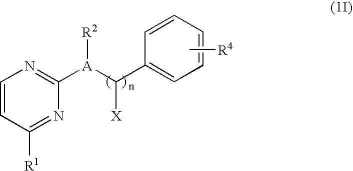

and pharmaceutically acceptable salts, hydrates, and solvates thereof,
wherein
R¹ is aryl, or heteroaryl, both optionally substituted by 1-7 R⁴ groups, provided that R⁴, when substituted on R¹, is not CO₂C₁-C₆alkyl;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the phenyl ring is optionally substituted with 1-5 independently selected R⁴ groups.

In another aspect, the invention provides compounds of Formula 1J:

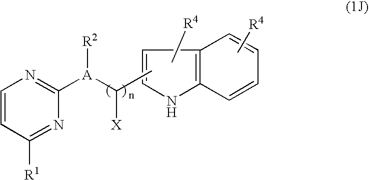

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, or CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
   wherein the indolyl ring is optionally substituted with 1-6 independently selected R⁴ groups.

In another aspect, the invention provides compounds of Formula 1K:

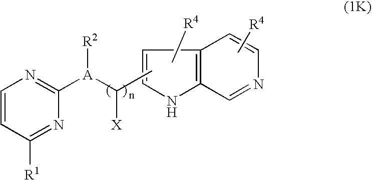

and pharmaceutically acceptable salts, hydrates, and solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, C(O)O C₁-C₆ alkyl, CN, OH, cycloalkyl, CONH₂, aryl, heteroaryl, COaryl, or trifluoroacetyl;
   wherein the pyrrolpyridinyl ring is optionally substituted with 1-5 independently selected R⁴ groups.

In another aspect, the invention provides methods of synthesizing compounds of the invention comprising:

reacting a compound of the Formula (2):

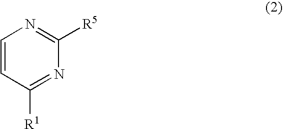

wherein

R¹ is aryl or heteroaryl, both optionally substituted by 1-7 R⁴ groups;

each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl; and,

R⁵ is halogen;

with a compound of Formula 3:

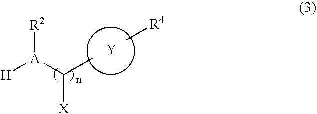

wherein

A is CH, N, O, S, SO or SO₂;

n is 1-4;
R²H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;

each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;

Y is imidazolyl, triazolyl, pyrazolyl, pyrrolyl, pyridinyl, pyrimidinyl, pyridizinyl, pyrazinyl, phenyl, indolyl, pyrolepyridinyl, all optionally substituted by 1-6 R⁴ groups; and

each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;

under conditions effective to substitute R⁵ with the compound of Formula (3) thereby providing a compound having the Formula (1):

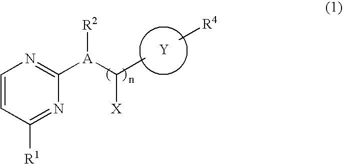

and pharmaceutically acceptable salts, hydrates, and solvates thereof,
wherein

R¹ is aryl, or heteroaryl, both optionally substituted by 1-7 independently selected R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 independently selected R⁴ groups except H;
Y is imidazolyl, triazolyl, pyrazolyl, pyrrolyl, pyridinyl, pyrimidinyl, pyridizinyl, pyrazinyl, phenyl, indolyl, pyrolepyridinyl, all optionally substituted by 1-6 R⁴ groups; and
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

In other aspects, the invention provides pharmaceutical compositions comprising compounds or pharmaceutically acceptable salts of compounds of Formula (1), Formula (1A), Formula (1B), Formula (1C), Formula (1D), Formula (1E), Formula (1F), Formula (1G), Formula (1H), Formula (1I), Formula (1J), and Formula (1K), or pharmaceutically acceptable salts, hydrates, or solvates thereof, and a pharmaceutically acceptable carrier.

In one aspect, the compounds or pharmaceutically acceptable salts of the compounds of Formula (1), Formula (1A), Formula (1B), Formula (1C), Formula (1D), Formula (1E), Formula (1F), Formula (1G), Formula (1H), Formula (1I), Formula (1J), and Formula (1K) are useful as canonical Wnt-β-catenin cellular messaging system agonists.

In some embodiments, the invention provides methods for treating a canonical Wnt-β-catenin cellular messaging system related disorder, comprising administering to a mammal in need thereof a compound or a pharmaceutically acceptable salt of a compound of Formula (1), Formula (1A), Formula (1B), Formula (1C), Formula (1D), Formula (1E), Formula (1F), Formula (1G), Formula (1H), Formula (1I), Formula (1J), and Formula (1K) in an amount effective to treat a canonical Wnt-β-catenin cellular messaging system related disorder.

### DETAILED DESCRIPTION OF THE INVENTION

The following definitions are used in connection with the heteroaryl/aryl pyrimidine analogs of the present invention:

“Alkyl” refers to a hydrocarbon chain that may be a straight chain or branched chain, containing the indicated number of carbon atoms. For example, C₁-C₆ indicates that the group may have from 1, 2, 3, 4, 5 or 6 carbon atoms in it.

“Aryl” refers to cyclic aromatic carbon ring systems made from 6 to 18 carbons. Examples of an aryl group include, but are not limited to, phenyl, napthyl, anthracenyl, tetracenyl, and phenanthrenyl. An aryl group can be unsubstituted or substituted with one or more of the following groups: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroaryl” refers to mono and bicyclic aromatic groups of 4 to 10 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Arylalkyl” refers to an aryl group with at least one alkyl substitution. Examples of arylalkyl include, but are not limited to, toluenyl, phenylethyl, xylenyl, phenylbutyl, phenylpentyl, and ethylnapthyl. An arylalkyl group can be unsubstituted or substituted with one or more of the following groups: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroarylalkyl” refers to a heteroaryl group with at least one alkyl substitution. A heteroarylalkyl group can be unsubstituted or substituted with one or more of the following: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₃-C₆ alkenyl” refers to a straight or branched chain unsaturated hydrocarbon containing 3-6 carbon atoms and at least one double bond. Examples of a C₃-C₆ alkenyl group include, but are not limited to, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” refers to a straight or branched chain saturated or unsaturated hydrocarbon containing 1-6 carbon atoms and at least one oxygen atom. Examples of a C₁-C₆ alkoxy include, but are not limited to, methoxy, ethoxy, isopropoxy, butoxy, n-pentoxy, isopentoxy, neopentoxy, and hexoxy

A “subject” is a mammal, e.g., a human, mouse, rat, guinea pig, dog, cat, horse, cow, pig, or non-human primate, such as a monkey, chimpanzee, baboon or rhesus.

The invention also provides pharmaceutical compositions comprising an effective amount of a heteroaryl/aryl pyrimidine analog and a pharmaceutically acceptable carrier. The invention provides a heteroaryl/aryl pyrimidine analog when provided as a pharmaceutically acceptable prodrug, hydrated salt, such as a pharmaceutically acceptable salt, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection an heteroaryl/aryl pyrimidine analog is an amount effective for treating or preventing a disease associated with the canonical Wnt-β-catenin cellular messaging system.

The following abbreviations are used herein and have the indicated definitions: ACN is acetonitrile, HOAc is acetic acid, n-BuLi is normal butyl lithium, DDQ is 2,3-dicyano-5,6-dichloro-parabenzoquinone, DIEA is diisopropylethylamine, DMF is N,N-dimethylformamide, DMSO is dimethylsulfoxide, EtOAc is ethyl acetate, EtOH is ethanol, FBS is fetal bovine serum, HPLC is high pressure liquid chromatography, I—Pr₂NEt is diisopropylethylamine, MeCN is acetonitrile, MeOH is methanol, MS is mass spectrometry, NEt₃ is triethylamine, NMP is N-methyl-2-pyrrolidone, NMR is nuclear magnetic resonance, PBS is phosphate-buffered saline (pH 7.4), RPMI is Roswell Park Memorial Institute, T-BuOK is potassium tert-Butoxide, THF is tetrahydrofuran, TFA is trifluoroacetic acid, and TLC is thin-layer chromatography, VLUX is a device for measuring luminescence.

### The Heteroaryl/Aryl Pyrimidine Analog of Formula 1

The present invention provides heteroaryl/aryl pyrimidine analogs according to Formula (1) below:

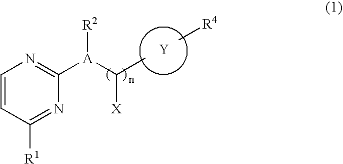

and pharmaceutically acceptable salts, hydrates, and solvates thereof,
wherein
R¹, R², A, X, Y, n, and R⁴ are as defined above for the compounds of Formula (1).

In one embodiment, Y is imidazolyl.

In one embodiment, Y is triazolyl.

In one embodiment, Y is pyrazolyl.

In one embodiment, Y is pyrrolyl.

In one embodiment, Y is pyridinyl.

In one embodiment, Y is pyrimidinyl.

In one embodiment, Y is pyridizinyl.

In one embodiment, Y is pyrazinyl.

In one embodiment, Y is phenyl.

In one embodiment, Y is indolyl.

In one embodiment, Y is pyrolepyridinyl.

In one embodiment, R¹ is optionally substituted aryl.

In one embodiment, R¹ is optionally substituted heteroaryl.

In one embodiment, A is nitrogen.

The invention also relates to compounds of Formula (1A):

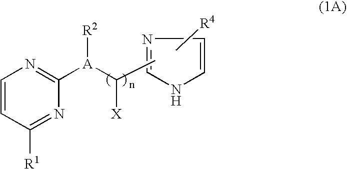

and pharmaceutically acceptable salts, hydrates, and solvates thereof,
wherein

R¹, R², A, X, n, and R⁴ are as defined above for the compounds of Formula (1A).

In one embodiment, the compound is (1A-1):

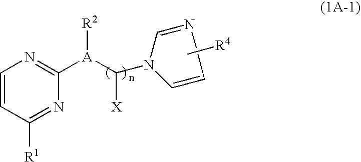

In one embodiment, the compound is (1A-2):

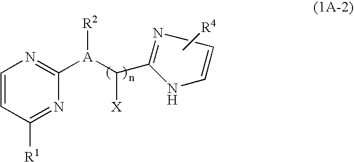

In one embodiment, the compound is (1A-3):

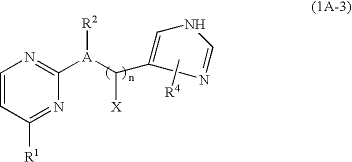

In one embodiment, the compound is (1A-4):

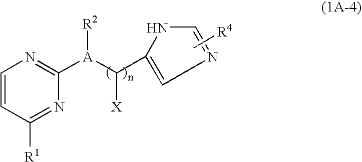

Illustrative compounds of Formula 1A are exemplified by the following compounds:

| Example. | Compound Name |
|---|---|
| 1 | N-[3-(1H-imidazol-1-yl)propyl]-4-(4-pyridinyl)-2-pyrimidinamine; |
| 2 | N-[3-(1H-imidazol-1-yl)propyl]-4-(3-pyridinyl)-2-pyrimidinamine; |
| 3 | N-[3-(1H-imidazol-1-yl)propyl]-4-(3-nitrophenyl)-2-p+C10yrimidinamine; |
| 6 | N-[2-(1H-imidazol-4-yl)ethyl]-4-(2-naphthyl)pyrimidin-2-amine; |
| 7 | N-[2-(1H-imidazol-4-yl)ethyl]-4-pyridin-3-ylpyrimidin-2-amine; |
| 8 | (2S)-3-(1H-imidazol-4-yl)-2-{[4-(2-naphthyl)pyrimidin-2-yl]amino}propan-1-ol; |
| 17 | 4-(1-benzothien-2-yl)-N-[2-(1H-imidazol-4-yl)ethyl]pyrimidin-2-amine; |
| 23 | N-benzyl-N-[4-(2-naphthyl)pyrimidin-2-yl]histidinamide; |
| 24 | N-[2-(4-methoxyphenyl)ethyl]-N-[4-(2-naphthyl)pyrimidin-2-yl]histidinamide; |
| 25 | ethyl [4-((2S)-3-hydroxy-2-{[4-(2-naphthyl)pyrimidin-2-yl]amino}propyl)-1H-imidazol-1- |
| | yl]acetate; |
| 26 | (2S)-2-{[4-(2-naphthyl)pyrimidin-2-yl]amino}-3-(1-prop-2-yn-1-yl-1H-imidazol-4- |
| | yl)propan-1-ol; |
| 27 | [4-((2S)-3-hydroxy-2-{[4-(2-naphthyl)pyrimidin-2-yl]amino}propyl)-1H-imidazol-1- |
| | yl]acetonitrile; |
| 28 | 5-[4-((2S)-3-hydroxy-2-{[4-(2-naphthyl)pyrimidin-2-yl]amino}propyl)-1H-imidazol-1- |
| | yl]pentanenitrile; |
| 29 | 2-[4-((2S)-3-hydroxy-2-{[4-(2-naphthyl)pyrimidin-2-yl]amino}propyl)-1H-imidazol-1- |
| | yl]acetamide; |
| 30 | (2S)-3-[1-(3,5-difluorobenzyl)-1H-imidazol-4-yl]-2-{[4-(2-naphthyl)pyrimidin-2- |
| | yl]amino}propan-1-ol; |
| 31 | 1,1,1-trifluoro-3-[4-((2S)-3-hydroxy-2-{[4-(2-naphthyl)pyrimidin-2-yl]amino}propyl)-1H- |
| | imidazol-1-yl]acetone; |
| 32 | 1-(3-{[4-((2S)-3-hydroxy-2-{[4-(2-naphthyl)pyrimidin-2-yl]amino}propyl)-1H-imidazol-1- |
| | yl]methyl}-4-methoxyphenyl)ethanone; |
| 33 | (2S)-2-{[4-(1-benzothien-2-yl)pyrimidin-2-yl]amino}-3-(1-prop-2-yn-1-yl-1H-imidazol-4- |
| | yl)propan-1-ol; |
| 34 | [4-((2S)-2-{[4-(1-benzothien-2-yl)pyrimidin-2-yl]amino}-3-hydroxypropyl)-1H-imidazol- |
| | 1-yl]acetonitrile; |
| 35 | 5-[4-((2S)-2-{[4-(1-benzothien-2-yl)pyrimidin-2-yl]amino}-3-hydroxypropyl)-1H- |
| | imidazol-1-yl]pentanenitrile; |
| 36 | 2-[4-((2S)-2-{[4-(1-benzothien-2-yl)pyrimidin-2-yl]amino}-3-hydroxypropyl)-1H- |
| | imidazol-1-yl]acetamide; |
| 37 | (2S)-2-{[4-(1-benzothien-2-yl)pyrimidin-2-yl]amino}-3-[1-(3,5-difluorobenzyl)-1H- |
| | imidazol-4-yl]propan-1-ol; |
| 38 | 1-(3-{[4-((2S)-2-{[4-(1-benzothien-2-yl)pyrimidin-2-yl]amino}-3-hydroxypropyl)-1H- |
| | imidazol-1-yl]methyl}-4-methoxyphenyl)ethanone; |
| 39 | 1-[4-((2S)-2-{[4-(1-benzothien-2-yl)pyrimidin-2-yl]amino}-3-hydroxypropyl)-1H- |
| | imidazol-1-yl]butan-2-ol; |
| 40 | [4-((2S)-3-hydroxy-2-{[4-(5-methyl-2-thienyl)pyrimidin-2-yl]amino}propyl)-1H-imidazol- |
| | 1-yl]acetonitrile; |
| 41 | 5-[4-((2S)-3-hydroxy-2-{[4-(5-methyl-2-thienyl)pyrimidin-2-yl]amino}propyl)-1H- |
| | imidazol-1-yl]pentanenitrile; |
| 42 | (2S)-3-[1-(4-methylpent-3-en-1-yl)-1H-imidazol-4-yl]-2-{[4-(5-methyl-2- |
| | thienyl)pyrimidin-2-yl]amino}propan-1-ol; |
| 43 | (2S)-3-[1-(3-cyclohexylpropyl)-1H-imidazol-4-yl]-2-{[4-(5-methyl-2-thienyl)pyrimidin-2- |
| | yl]amino}propan-1-ol; |
| 44 | (2S)-3-[1-(3,5-difluorobenzyl)-1H-imidazol-4-yl]-2-{[4-(5-methyl-2-thienyl)pyrimidin-2- |
| | yl]amino}propan-1-ol; |
| 45 | 1-(3-{[4-((2S)-3-hydroxy-2-{[4-(5-methyl-2-thienyl)pyrimidin-2-yl]amino}propyl)-1H- |
| | imidazol-1-yl]methyl}-4-methoxyphenyl)ethanone; |
| 46 | (2S)-3-{1-[(2,6-dichloropyridin-4-yl)methyl]-1H-imidazol-4-yl}-2-{[4-(5-methyl-2- |
| | thienyl)pyrimidin-2-yl]amino}propan-1-ol; |
| 47 | 2-[4-((2S)-3-hydroxy-2-{[4-(5-methyl-2-thienyl)pyrimidin-2-yl]amino}propyl)-1H- |
| | imidazol-1-yl]-1-[4-(trifluoromethyl)phenyl]ethanone; |
| 48 | N-[2-ethoxy-1-(1H-imidazol-4-ylmethyl)ethyl]-4-(2-naphthyl)pyrimidin-2-amine; |
| 49 | N-[2-(cyclohexylmethoxy)-1-(1H-imidazol-4-ylmethyl)ethyl]-4-(2-naphthyl)pyrimidin-2- |
| | amine; |
| 50 | N-[2-[(5-tert-butyl-1,2,4-oxadiazol-3-yl)methoxy]-1-(1H-imidazol-4-ylmethyl)ethyl]-4-(2- |
| | naphthyl)pyrimidin-2-amine; |
| 51 | N-[2-{[2-fluoro-3-(trifluoromethyl)benzyl]oxy}-1-(1H-imidazol-4-ylmethyl)ethyl]-4-(2- |
| | naphthyl)pyrimidin-2-amine; |
| 52 | N-[2-(1H-imidazol-4-yl)-1-({[5-(2-methoxyphenyl)-1,2,4-oxadiazol-3- |
| | yl]methoxy}methyl)ethyl]-4-(2-naphthyl)pyrimidin-2-amine; |
| 53 | 4-(1-benzothien-2-yl)-N-{2-(1H-imidazol-4-yl)-1-[(prop-2-yn-1- |
| | yloxy)methyl]ethyl}pyrimidin-2-amine; |
| 54 | 4-(1-benzothien-2-yl)-N-[2-[(5-tert-butyl-1,2,4-oxadiazol-3-yl)methoxy]-1-(1H-imidazol- |
| | 4-ylmethyl)ethyl]pyrimidin-2-amine; |
| 55 | 2-{[2-{[4-(1-benzothien-2-yl)pyrimidin-2-yl]amino}-3-(1H-imidazol-4- |
| | yl)propoxy]methyl}benzonitrile; and |
| 56 | N-[2-[(5-tert-butyl-1,2,4-oxadiazol-3-yl)methoxy]-1-(1H-imidazol-4-ylmethyl)ethyl]-4-(2- |
| | thienyl)pyrimidin-2-amine. |

The invention also relates to compounds of Formula (1B):

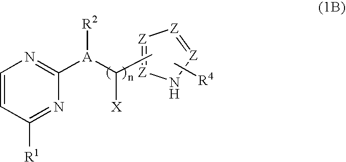

and pharmaceutically acceptable salts, hydrates, and solvates thereof,
wherein

R¹, R², A, X, n, Z, and R⁴ are as defined above for the compounds of Formula (1B).

In one embodiment, the compound is (1B-1):

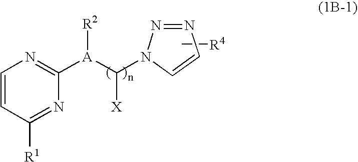

In one embodiment, the compound is (1B-2):

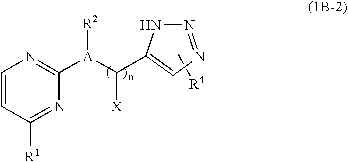

In one embodiment, the compound is (1B-3):

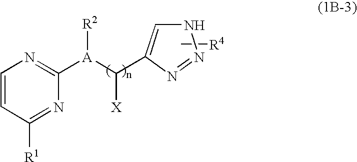

In one embodiment, the compound is (1B-4):

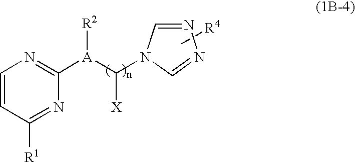

In one embodiment, the compound is (1B-5):

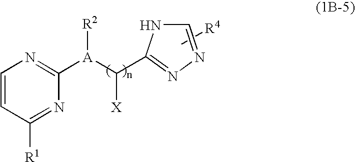

In one embodiment, the compound is (1B-6):

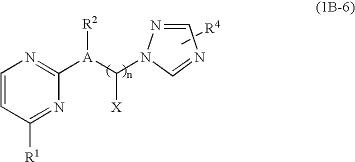

In one embodiment, the compound is (1B-7):

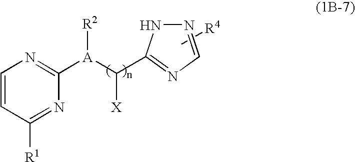

In one embodiment, the compound is (1B-8):

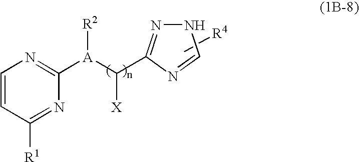

In one embodiment, the compound is (1B-9):

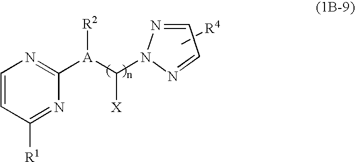

In one embodiment, the compound is (1B-10):

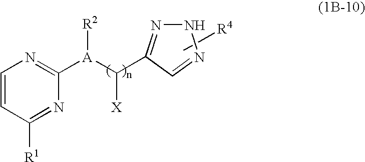

The invention also relates to compounds of Formula (1C):

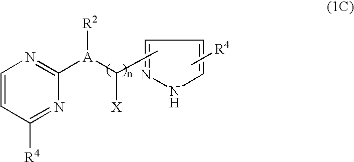

and pharmaceutically acceptable salts, hydrates, and solvates thereof,
wherein

R¹, R², A, X, n, and R⁴ are as defined above for the compounds of Formula (1C).

In one embodiment, the compound is (1C-1):

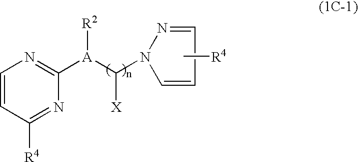

In one embodiment, the compound is (1C-2):

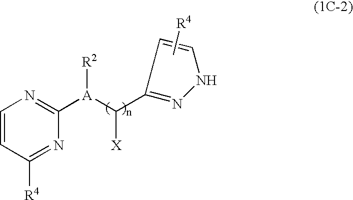

In one embodiment, the compound is (1C-3):

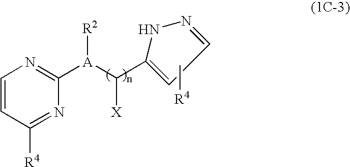

In one embodiment, the compound is (1C-4):

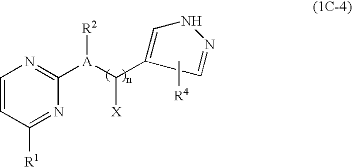

Illustrative compounds of Formula 1C are exemplified by the following compounds:

| Example | Compound Name |
|---|---|
| 5 | 4-{3-[(4-pyridin-2-ylpyrimidin-2-yl)amino]propyl}- |
| | 2,4-dihydro-3H-pyrazol-3-one; |
| 9 | N-[3-(3,5-dimethyl-1H-pyrazol-1-yl)propyl]-4- |
| | (2-naphthyl)pyrimidin-2-amine; and |
| 10 | N-[3-(3,5-dimethyl-1H-pyrazol-1-yl)propyl]-4- |
| | pyridin-4-ylpyrimidin-2-amine. |

The invention also relates to compounds of Formula (1D):

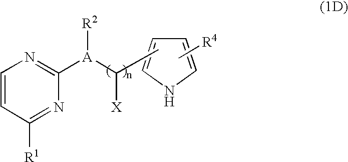

and pharmaceutically acceptable salts, hydrates, and solvates thereof,
wherein

R¹, R², A, X, n, and R⁴ are as defined above for the compounds of Formula (ID).

In one embodiment, the compound is (1D-1):

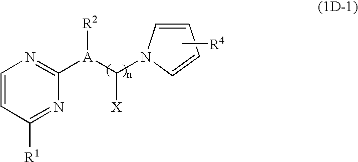

In one embodiment, the compound is (1D-2):

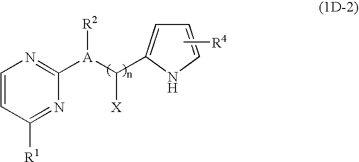

In one embodiment, the compound is (1D-3):

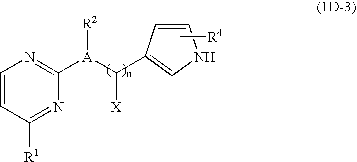

The invention also relates to compounds of Formula (1E):

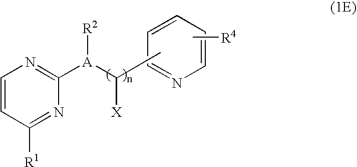

and pharmaceutically acceptable salts, hydrates, and solvates thereof,
wherein

R¹, R², A, X, n, and R⁴ are as defined above for the compounds of Formula (1E).

In one embodiment, the compound is (1E-1):

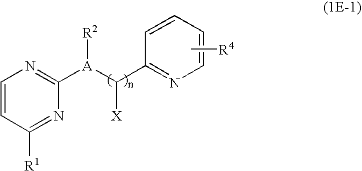

In one embodiment, the compound is (1E-2):

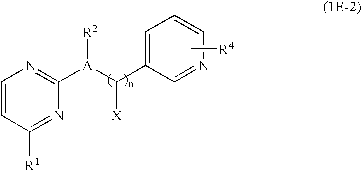

In one embodiment, the compound is (1E-3):

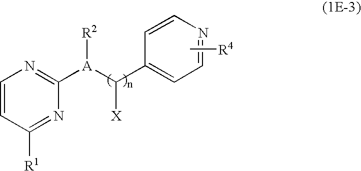

Illustrative compounds of Formula 1E are exemplified by the following compounds:

| Example | Compound Name |
|---|---|
| 4 | 4-pyrazin-2-yl-N-(2-pyridin-2-ylethyl)pyrimidin-2-amine; |
| 13 | 4-pyridin-3-yl-N-(2-pyridin-2-ylethyl)pyrimidin-2-amine; |
| 14 | 4-pyridin-3-yl-N-(2-pyridin-4-ylethyl)pyrimidin-2-amine; |
| 16 | 4-pyridin-4-yl-N-(2-pyridin-4-ylethyl)pyrimidin-2-amine; |
| 18 | 4-(1-benzothien-2-yl)-N-(2-pyridin-2-ylethyl)pyrimidin-2- |
| | amine; |
| 20 | 4-(1-naphthyl)-N-(2-pyridin-2-ylethyl)pyrimidin-2-amine; |
| 21 | 4-(5-bromothien-2-yl)-N-(2-pyridin-2-ylethyl)pyrimidin-2- |
| | amine; |
| 22 | 4-(5-bromothien-2-yl)-N-(2-pyridin-4-ylethyl)pyrimidin-2- |
| | amine; |
| 63 | 4-(naphthalen-2-yl)-N-(pyridin-3-ylmethyl)pyrimidin-2-amine; |
| 64 | N-(2-(pyridin-3-yl)ethyl)-4-(pyridin-4-yl)pyrimidin-2- |
| | amine; and |
| 65 | N-(2-(pyridin-3-yl)ethyl)-4-(thiophen-2-yl)pyrimidin-2-amine. |

The invention also relates to compounds of Formula (1F):

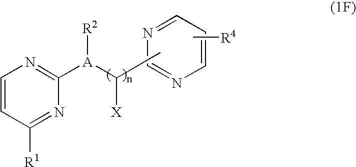

and pharmaceutically acceptable salts, hydrates, and solvates thereof,
wherein

R¹, R², A, X, n, and R⁴ are as defined above for the compounds of Formula (1F).

In one embodiment, the compound is (1F-1):

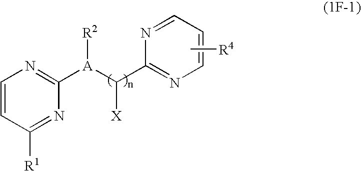

In one embodiment, the compound is (1F-2):

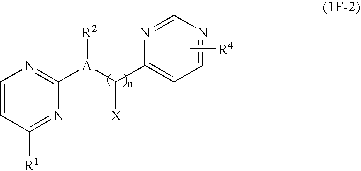

In one embodiment, the compound is (1F-3):

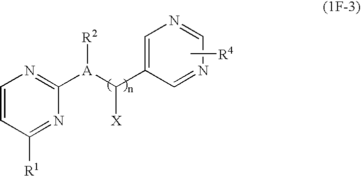

The invention also relates to compounds of Formula (1G):

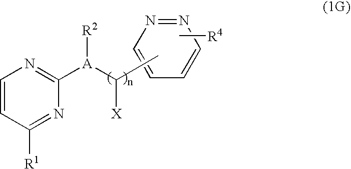

and pharmaceutically acceptable salts, hydrates, and solvates thereof,
wherein

R¹, R², A, X, n, and R⁴ are as defined above for the compounds of Formula (1G).

In one embodiment, the compound is (1G-1):

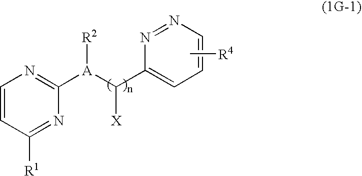

In one embodiment, the compound is (1G-2):

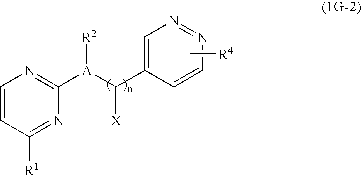

The invention also relates to compounds of Formula (1H):

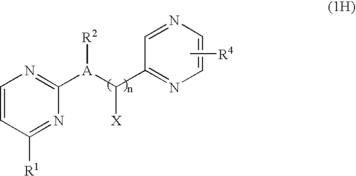

and pharmaceutically acceptable salts, hydrates, and solvates thereof,
wherein

R¹, R², A, X, n, and R⁴ are as defined above for the compounds of Formula (1H).

The invention also relates to compounds of Formula (1I):

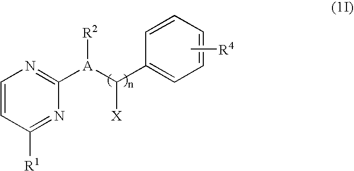

and pharmaceutically acceptable salts, hydrates, and solvates thereof,
wherein

R¹, R², A, X, n, and R⁴ are as defined above for the compounds of Formula (1I).

Illustrative compounds of Formula II are exemplified by the following compound:

| | |
|---|---|
| 59 | (2R)-2-{[4-(2-naphthyl)pyrimidin-2-yl]amino}-3-phenylpropan-1-ol |

The invention also relates to compounds of Formula (1J):

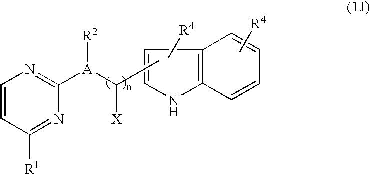

and pharmaceutically acceptable salts, hydrates, and solvates thereof,
wherein

R¹, R², A, X, n, and R⁴ are as defined above for the compounds of Formula (1J).

In one embodiment, the compound is (1J-1):

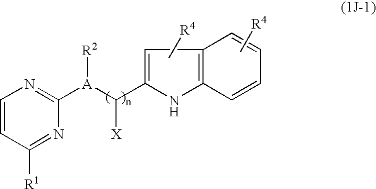

In one embodiment, the compound is (1J-2):

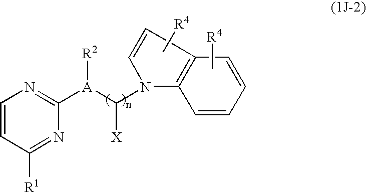

In one embodiment, the compound is (1J-3):

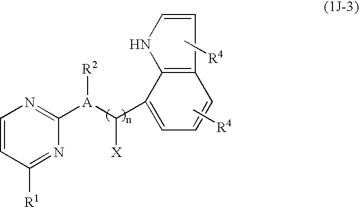

In one embodiment, the compound is (1J-4):

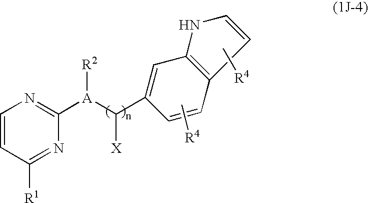

In one embodiment, the compound is (1J-5):

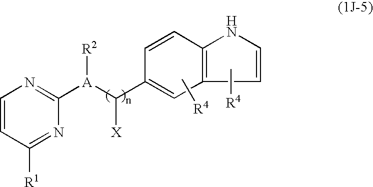

In one embodiment, the compound is (1J-6):

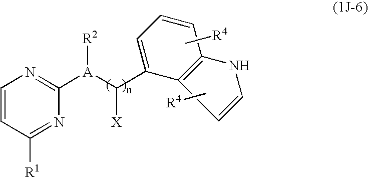

In one embodiment, the compound is (1J-7):

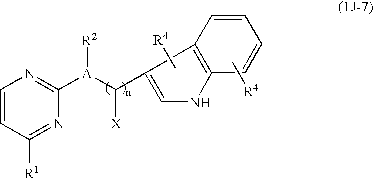

Illustrative compounds of Formula 1J are exemplified by the following compounds:

| Example | Compound Name |
|---|---|
| 11 | N-[2-(1H-indol-3-yl)ethyl]-4-pyridin-3-ylpyrimidin-2-amine; |
| 12 | 2-methyl-3-{2-[(4-pyridin-3-ylpyrimidin-2- |
| | yl)amino]ethyl}-1H-indol-5-ol; |
| 15 | N-[2-(1H-indol-3-yl)ethyl]-4-pyridin-4-ylpyrimidin- |
| | 2-amine; |
| 57 | (2S)-3-(1H-indol-3-yl)-2-{[4-(2-thienyl)pyrimidin- |
| | 2-yl]amino}propan-1-ol; |
| 58 | (2R)-3-(1H-indol-3-yl)-2-{[4-(2-naphthyl)pyrimidin- |
| | 2-yl]amino}propan-1-ol; |
| 60 | (2S)-3-(1H-indol-3-yl)-2-{[4-(2-naphthyl)pyrimidin- |
| | 2-yl]amino}propan-1-ol; |
| 61 | N-[2-(1H-indol-3-yl)ethyl]-4-(2-naphthyl)pyrimidin- |
| | 2-amine; and |
| 62 | (2S)-3-(1H-indol-3-yl)-2-[(4-pyridin-4-ylpyrimidin-2- |
| | yl)amino]propan-1-ol. |

The invention also relates to compounds of Formula (1K):

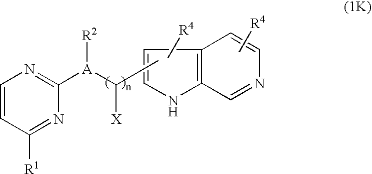

and pharmaceutically acceptable salts, hydrates, and solvates thereof,
wherein

R¹, R², A, X, n, and R⁴ are as defined above for the compounds of Formula (1K).

In one embodiment, the compound is (1K-1):

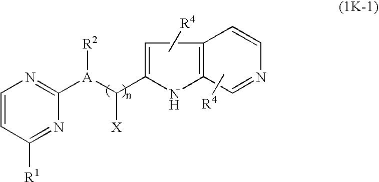

In one embodiment, the compound is (1K-2):

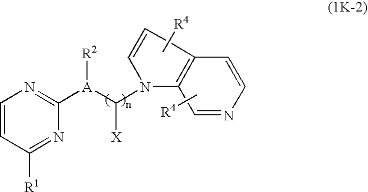

In one embodiment, the compound is (1K-3):

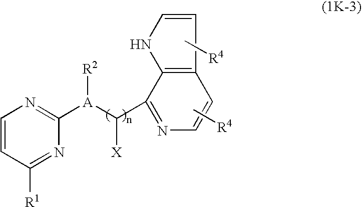

In one embodiment, the compound is (1K-4):

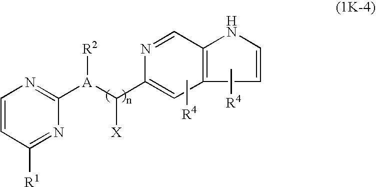

In one embodiment, the compound is (1K-5):

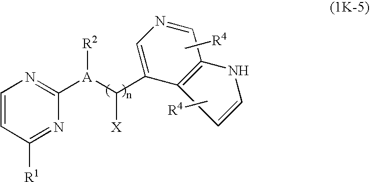

In one embodiment, the compound is (1K-6):

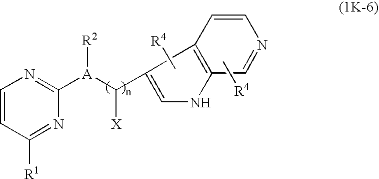

Illustrative compounds of Formula 1K are exemplified by the following compound:

| | |
|---|---|
| 19 | 4-(1-naphthyl)-N-[2-(1H-pyrrolo[2,3-c]pyridin- |
| | 3-yl)ethyl]pyrimidin-2-amine |

### Methods for Using Heteroaryl/Aryl Pyrimidine Analogs

The heteroaryl/aryl pyrimidine analogs of the present invention exhibit agonism of the canonical Wnt-β-catenin cellular messaging system and, therefore, can be utilized in order to inhibit abnormal cell growth and/or encourage healthy cell regeneration or healthy cell growth. Thus, the heteroaryl/aryl pyrimidine analogs are effective in the treatment of disorders of the canonical Wnt-β-catenin cellular messaging system including, bone disorders, cancer, and Alzheimer's disease. In particular, the heteroaryl/aryl pyrimidine analogs of the present invention possess excellent cancer cell growth inhibiting effects and are effective in treating cancers. Types of cancers that can be treated include but are not limited to solid cancers and malignant lymphomas, and also, leukemia, skin cancer, bladder cancer, breast cancer, uterus cancer, ovary cancer, prostate cancer, lung cancer, colon cancer, pancreas cancer, renal cancer, gastric cancer, brain tumor.

### Therapeutic Administration

When administered to a subject, the heteroaryl/aryl pyrimidine analogs or pharmaceutically acceptable salts of the heteroaryl/aryl pyrimidine analogs can be administered neat or as a component of a pharmaceutical composition that comprises a physiologically acceptable carrier or vehicle. A pharmaceutical composition of the invention can be prepared using a method comprising admixing the heteroaryl/aryl pyrimidine analogs or a pharmaceutically acceptable salt of the heteroaryl/aryl pyrimidine analogs and a physiologically acceptable carrier, excipient, or diluent. Admixing can be accomplished using methods well known for admixing a heteroaryl/aryl pyrimidine analog or a pharmaceutically acceptable salt of the heteroaryl/aryl pyrimidine analog and a physiologically acceptable carrier, exipient, or diluent.

The present compositions, comprising heteroaryl/aryl pyrimidine analogs or pharmaceutically acceptable salts of the heteroaryl/aryl pyrimidine analogs of the invention can be administered orally. The heteroaryl/aryl pyrimidine analogs or pharmaceutically acceptable salts of heteroaryl/aryl pyrimidine analogs of the invention can also be administered by any other convenient route, for example, by infusion or bolus injection, by absorption through epithelial or mucocutaneous linings (e.g., oral, rectal, vaginal, and intestinal mucosa) and can be administered together with another therapeutic agent. Administration can be systemic or local. Various known delivery systems, including encapsulation in liposomes, microparticles, microcapsules, and capsules, can be used.

Methods of administration include, but are not limited to, enteral or parenteral administration such as intradermal, intramuscular, intraperitoneal, intravascular (e.g., intravenous or intra-arterial), subcutaneous, intranasal, epidural, oral, sublingual, intracerebral, intravaginal, intra-articular, intrathecal, transdermal, rectal, by inhalation, or topical. In some instances, administration will result in release of the heteroaryl/aryl pyrimidine analog or a pharmaceutically acceptable salt of the heteroaryl/aryl pyrimidine analog into the bloodstream. The mode of administration is left to the discretion of the practitioner.

In one embodiment, the heteroaryl/aryl pyrimidine analog or a pharmaceutically acceptable salt of the heteroaryl/aryl pyrimidine analog is administered orally.

In another embodiment, the heteroaryl/aryl pyrimidine analog or a pharmaceutically acceptable salt of the heteroaryl/aryl pyrimidine analog is administered intravenously.

In another embodiment, the heteroaryl/aryl pyrimidine analog or a pharmaceutically acceptable salt of the heteroaryl/aryl pyrimidine analog can be administered locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, the heteroaryl/aryl pyrimidine analog or a pharmaceutically acceptable salt of the heteroaryl/aryl pyrimidine analog can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the heteroaryl/aryl pyrimidine analog or a pharmaceutically acceptable salt of the heteroaryl/aryl pyrimidine analog can be formulated as a suppository, with traditional binders and excipients such as triglycerides.

In another embodiment, the heteroaryl/aryl pyrimidine analog or a pharmaceutically acceptable salt of the heteroaryl/aryl pyrimidine analog can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989), the disclosure of which is herein incorporated by reference).

In yet another embodiment, the heteroaryl/aryl pyrimidine analog or a pharmaceutically acceptable salt of the heteroaryl/aryl pyrimidine analog can be delivered in a controlled-release system or sustained-release system (see, e.g., Goodson, in Medical Applications of Controlled Release, vol. 2, pp. 115-138 (1984), the disclosure of which is herein incorporated by reference). Other controlled or sustained-release systems discussed in the review by Langer, Science 249:1527-1533 (1990), the disclosure of which is herein incorporated by reference, can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989), the disclosures of which are herein incorporated by reference). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989), the disclosures of which are herein incorporated by reference).

In yet another embodiment, a controlled- or sustained-release system can be placed in proximity of a target of the heteroaryl/aryl pyrimidine analog or a pharmaceutically acceptable salt of the heteroaryl/aryl pyrimidine analog, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to a subject. The physiologically acceptable excipient should be stable under the conditions of manufacture and storage and should be preserved against the contaminating action of microorganisms. Water is a particularly useful excipient when the heteroaryl/aryl pyrimidine analog or a pharmaceutically acceptable salt of the heteroaryl/aryl pyrimidine analogs is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The heteroaryl/aryl pyrimidine analog or pharmaceutically acceptable salt of the heteroaryl/aryl pyrimidine analog of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995), the disclosure of which is herein incorporated by reference.

In one embodiment, the heteroaryl/aryl pyrimidine analog or a pharmaceutically acceptable salt of the heteroaryl/aryl pyrimidine analog is formulated in accordance with routine procedures as a composition adapted for oral administration to humans. Compositions for oral delivery can be in the form of, for example, tablets, lozenges, buccal forms, troches, aqueous or oily suspensions or solutions, granules, powders, emulsions, capsules, syrups, or elixirs. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided heteroaryl/aryl pyrimidine analog or pharmaceutically acceptable salt of the heteroaryl/aryl pyrimidine analog. In tablets, the heteroaryl/aryl pyrimidine analog or pharmaceutically acceptable salt of the heteroaryl/aryl pyrimidine analog is mixed with a carrier having the necessary compression properties in suitable proportions and compacted in the shape and size desired. The powders and tablets can contain up to about 99% of the heteroaryl/aryl pyrimidine analog or pharmaceutically acceptable salt of the heteroaryl/aryl pyrimidine analog.

Capsules may contain mixtures of the heteroaryl/aryl pyrimidine analogs or pharmaceutically acceptable salts of the heteroaryl/aryl pyrimidine analogs with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the heteroaryl/aryl pyrimidine analog or a pharmaceutically acceptable salt of the heteroaryl/aryl pyrimidine analog can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the heteroaryl/aryl pyrimidine analog or a pharmaceutically acceptable salt of the heteroaryl/aryl pyrimidine analog is to be administered by infusion, it can be dispensed, for example, with an infusion bottle containing sterile pharmaceutical grade water or saline. Where the heteroaryl/aryl pyrimidine analog or a pharmaceutically acceptable salt of the heteroaryl/aryl pyrimidine analog is administered by injection, an ampule of sterile water for injection or saline can be provided so that the ingredients can be mixed prior to administration.

In another embodiment, the heteroaryl/aryl pyrimidine analog or pharmaceutically acceptable salt of the heteroaryl/aryl pyrimidine analog can be administered transdermally through the use of a transdermal patch. Transdermal administrations include administrations across the surface of the body and the inner linings of the bodily passages including epithelial and mucosal tissues. Such administrations can be carried out using the present heteroaryl/aryl pyrimidine analogs or pharmaceutically acceptable salts of the heteroaryl/aryl pyrimidine analogs, in lotions, creams, foams, patches, suspensions, solutions, and suppositories (e.g., rectal or vaginal).

Transdermal administration can be accomplished through the use of a transdermal patch containing the heteroaryl/aryl pyrimidine analog or pharmaceutically acceptable salt of the heteroaryl/aryl pyrimidine analog and a carrier that is inert to the heteroaryl/aryl pyrimidine analog or pharmaceutically acceptable salt of the heteroaryl/aryl pyrimidine analog, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the heteroaryl/aryl pyrimidine analog or pharmaceutically acceptable salt of the heteroaryl/aryl pyrimidine analog into the blood stream, such as a semi-permeable membrane covering a reservoir containing the heteroaryl/aryl pyrimidine analog or pharmaceutically acceptable salt of the heteroaryl/aryl pyrimidine analog with or without a carrier, or a matrix containing the active ingredient.

The heteroaryl/aryl pyrimidine analogs or pharmaceutically acceptable salts of the heteroaryl/aryl pyrimidine analogs of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The heteroaryl/aryl pyrimidine analog or a pharmaceutically acceptable salt of the heteroaryl/aryl pyrimidine analog can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the heteroaryl/aryl pyrimidine analog or a pharmaceutically acceptable salt of the heteroaryl/aryl pyrimidine analog, and can thus reduce the occurrence of adverse side effects.

Controlled- or sustained-release compositions can initially release an amount of the heteroaryl/aryl pyrimidine analog or a pharmaceutically acceptable salt of the heteroaryl/aryl pyrimidine analog that promptly produces the desired therapeutic or prophylactic effect, and gradually and continually release other amounts of the heteroaryl/aryl pyrimidine analog or a pharmaceutically acceptable salt of the heteroaryl/aryl pyrimidine analog to maintain this level of therapeutic or prophylactic effect over an extended period of time. To maintain a constant level of the heteroaryl/aryl pyrimidine analog or a pharmaceutically acceptable salt of the heteroaryl/aryl pyrimidine analog in the body, the heteroaryl/aryl pyrimidine analog or a pharmaceutically acceptable salt of the heteroaryl/aryl pyrimidine analog can be released from the dosage form at a rate that will replace the amount of the heteroaryl/aryl pyrimidine analog or a pharmaceutically acceptable salt of the heteroaryl/aryl pyrimidine analog being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

In certain embodiments, the present invention is directed to prodrugs of the heteroaryl/aryl pyrimidine analogs or pharmaceutically acceptable salts of heteroaryl/aryl pyrimidine analogs of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975) the disclosures of which are herein incorporated by reference.

The amount of the heteroaryl/aryl pyrimidine analog or a pharmaceutically acceptable salt of the heteroaryl/aryl pyrimidine analog that is effective for treating or preventing a canonical Wnt-β-catenin cellular messaging system-related disorder can be determined using standard clinical techniques. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. An effective amount can range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day. Higher or lower effective amounts are also contemplated. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one heteroaryl/aryl pyrimidine analog or a pharmaceutically acceptable salt of the heteroaryl/aryl pyrimidine analog is administered, the effective dosage amounts correspond to the total amount administered.

The amount of the heteroaryl/aryl pyrimidine analog or a pharmaceutically acceptable salt of the heteroaryl/aryl pyrimidine analog that is effective for treating or preventing a canonical Wnt-β-catenin cellular messaging system-related disorder can range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, pre-filled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The heteroaryl/aryl pyrimidine analog or a pharmaceutically acceptable salt of the heteroaryl/aryl pyrimidine analog can be assayed in vitro or in vivo for the desired therapeutic or prophylactic activity prior to use in humans. Animal model systems can be used to demonstrate safety and efficacy.

The present methods for treating or preventing a canonical Wnt-β-catenin cellular messaging system-related disorder can further comprise administering another therapeutic agent to the subject being administered the heteroaryl/aryl pyrimidine analog or a pharmaceutically acceptable salt of the heteroaryl/aryl pyrimidine analog. In one embodiment, the other therapeutic agent is administered in an effective amount.

Effective amounts of the other therapeutic agents are well known to those skilled in the art. However, it is well within the skilled artisan's ability to determine the other therapeutic agent's optimal effective amount range. The heteroaryl/aryl pyrimidine analog or a pharmaceutically acceptable salt of the heteroaryl/aryl pyrimidine analog and the other therapeutic agent can act additively or, in one embodiment, synergistically. In one embodiment, of the invention, where another therapeutic agent is administered to an subject, the effective amount of the heteroaryl/aryl pyrimidine analog or a pharmaceutically acceptable salt of the heteroaryl/aryl pyrimidine analog is less than its effective amount would be where the other therapeutic agent is not administered. In this case, without being bound by theory, it is believed that the heteroaryl/aryl pyrimidine analog or a pharmaceutically acceptable salt of the heteroaryl/aryl pyrimidine analog and the other therapeutic agent act synergistically.

Suitable other therapeutic agents useful in the methods and compositions of the present invention include, but are not limited to cancer agents, Alzheimer's agents, bone disorder agents, osteoporosis agents, rheumatoid arthritis agents, osteoarthritis agents, and hormone replacement agents.

Suitable cancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

Other therapeutic agents useful in the methods and compositions of the present invention include, but are not limited to hydroxyzine, glatiramer acetate, interferon beta-1a, interferon beta-1b, mitoxantrone, and natalizumab.

Suitable Alzheimer's agents useful in the methods and compositions of the present invention include, but are not limited to donepezil, galantamine, memantine, niacin, rivastigmine, and tacrine.

Suitable bone disorder and/or osteoporosis agents useful in the methods and compositions of the present invention include, but are not limited to alendronate, bazedoxifene, calcitonin, clomifene, lasofoxifene, ormeloxifene, raloxifene, tamoxifen, and toremifene.

Suitable rheumatoid arthritis agents useful in the methods and compositions of the present invention include, but are not limited to abatacept, acetaminophen adalimumab, aspirin, auranofin, azathioprine, celecoxib, cyclophosphamide, cyclosporine, diclofenac, etanercept, hydroxychloroquine, ibuprofen, indomethacin, infliximab, ketoprofen, leflunomide, methotrexate, minocycline, nabumetone, naproxen, rituximab, and sulfasalazine.

Suitable osteoarthritis agents useful in the methods and compositions of the present invention include, but are not limited to acetaminophen, aspirin, celecoxib, cortisone, hyaluronic acid, ibuprofen, nabumetone, naproxen, rofecoxib, and valdecoxib.

Suitable hormone replacement therapy agents useful in the methods and compositions of the present invention include, but are not limited to estrogen, estradiol, medroxyprogesterone, norethindrone, and progesterone.

In one embodiment, the heteroaryl/aryl pyrimidine analog or a pharmaceutically acceptable salt of the heteroaryl/aryl pyrimidine analog is administered concurrently with another therapeutic agent.

In one embodiment, a composition comprising an effective amount of the heteroaryl/aryl pyrimidine analog or a pharmaceutically acceptable salt of the heteroaryl/aryl pyrimidine analog and an effective amount of another therapeutic agent within the same composition can be administered.

In another embodiment, a composition comprising an effective amount of the heteroaryl/aryl pyrimidine analog or a pharmaceutically acceptable salt of the heteroaryl/aryl pyrimidine analog and a separate composition comprising an effective amount of another therapeutic agent can be concurrently administered.

In another embodiment, an effective amount of the heteroaryl/aryl pyrimidine analog or a pharmaceutically acceptable salt of the heteroaryl/aryl pyrimidine analog is administered prior to or subsequent to administration of an effective amount of another therapeutic agent. In this embodiment, the heteroaryl/aryl pyrimidine analog or a pharmaceutically acceptable salt of the heteroaryl/aryl pyrimidine analog is administered while the other therapeutic agent exerts its therapeutic effect, or the other therapeutic agent is administered while the heteroaryl/aryl pyrimidine analog or a pharmaceutically acceptable salt of the heteroaryl/aryl pyrimidine analog exerts its preventative or therapeutic effect for treating or preventing a canonical Wnt-β-catenin cellular messaging system-related disorder.

In another embodiment, the pharmaceutically acceptable carrier is suitable for oral administration and the composition comprises an oral dosage form.

The heteroaryl/aryl pyrimidine analogs and pharmaceutically acceptable salts of heteroaryl/aryl pyrimidine analogs can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

Methods useful for making the heteroaryl/aryl pyrimidine analogs are set forth in the Examples below and generalized in Schemes.

### Methods of Making Heteroaryl/Aryl Pyrimidine Analogs

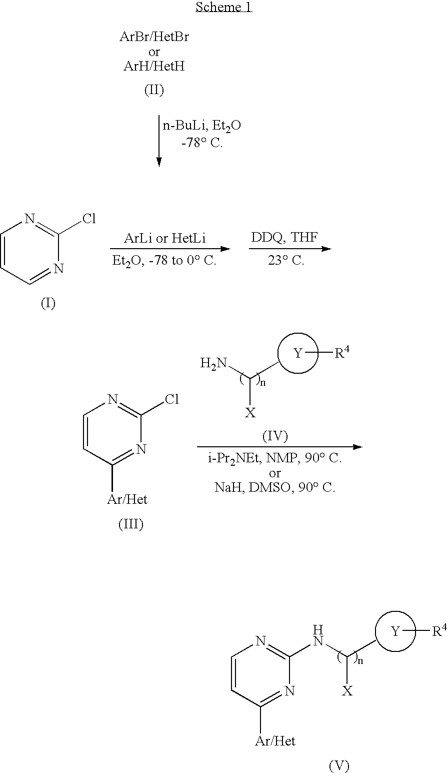

wherein R⁴, X, and n are as defined above for the heteroaryl/aryl pyrimidine analogs of Formula (1), Formula (1A), Formula (1B), Formula (1C), Formula (1D), Formula (1E), Formula (1F), Formula (1G), Formula (1H), Formula (1I), Formula (1J), or Formula (1K).

As shown in scheme 1, a compound of formula (V) may be prepared by reacting 2-chloropyrimidine (I) with an aryl or an heteroaryllithium, prepared by reacting an aryl bromide/heteroaryl bromide (II) with a strong base such as n-BuLi, MeLi or PhLi or via deprotonation of the aryl/heteroaryl (II) with a strong base such as n-BuLi, MeLi, PhLi, LDA, or LiN(TMS)₂, followed by oxidation with DDQ to give 4-aryl/heteroaryl-2-chloropyrimidine (III) according to the procedures of Czarny and Harden. (Strekowski, L et al., J. Heterocyclic. Chem. 1990, 27, 1393, and Harden D. B. et al., J. Org. Chem. 1988, 53, 4137), the disclosure of which is herein incorporated by reference. A subsequent reaction with amine (IV) in a polar aprotic solvent (e.g. NMP, DMSO, DMF, THF, or pyridine) and a base (e.g. iPr₂NEt, Et₃N, NaH, KH, K₂CO₃, Na₂CO₃, NaHCO₃ or t-BuOK) provides (V).

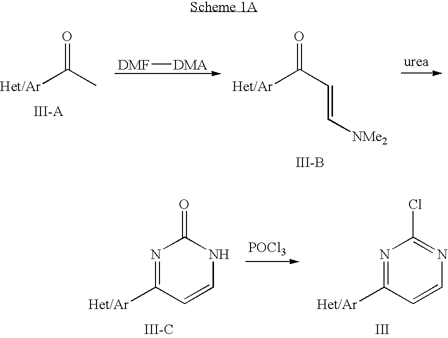

Alternatively as shown in Scheme 1A, the 4-aryl-2-chloropyrimidine intermediate (III) can be prepared by treating the corresponding arylacetyl compound (III-A) with DMF dimethylacetyl to provide the vinylogous amide (III-B). This is further treated with urea and the pyrimidinone product (III-C) is converted to the chloride III after refluxing in phosphorous oxychloride for several hours (see, e.g., WO 2005/049581, the disclosure of which is herein incorporated by reference).

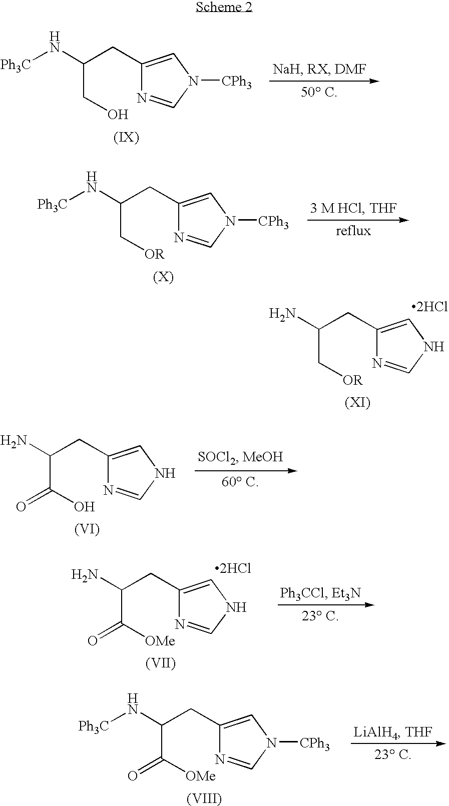

Amines (IV) can be purchased commercially or prepared via the procedure in Scheme 2 (Kovalainen et al. J. Med. Chem. 1999, 42, 1193, the disclosure of which is herein incorporated by reference). For example, histidine (VI) may be converted to its corresponding methyl ester (VII) via the action of a protic acid (e.g. HCl, H₂SO₄, HBr). Protection of the 1° and 2° amines may be accomplished using Ph₃CCl, an organic base such as Et₃N, i-Pr₂NEt, or pyridine to give (VIII). Reduction of the carboxylic acid to the 1° alcohol may be accomplished using LiAlH₄ in an ethereal solvent (e.g. THF, Et₂O, DME). O-alkylation of (IX) may be carried out using alkyl halides, benzyl halides or heteroaryl halides, using a strong base such as NaH, KH or using t-BuOK in a polar aprotic solvent (e.g. DMF, DMSO, NMP, MeCN, THF). Deprotection of the Ph₃C groups using a protic acid (e.g. HCl, HBr, H₂SO₄) gives the desired amines (XI).

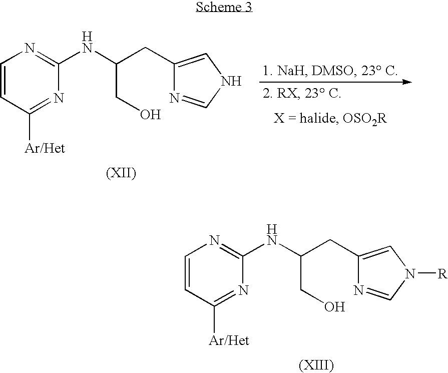

Target compounds of structure (XIII) may be prepared by reacting histidinol analog (XII), prepared via the procedure outlined in Scheme 1 above, with a strong base such as NaH, KH, or t-BuOK in a polar aprotic solvent (e.g. DMSO, NMP, DMF, MeCN); followed by treatment with the appropriate alkyl or benzyl halide/sulfonate at 23° C.

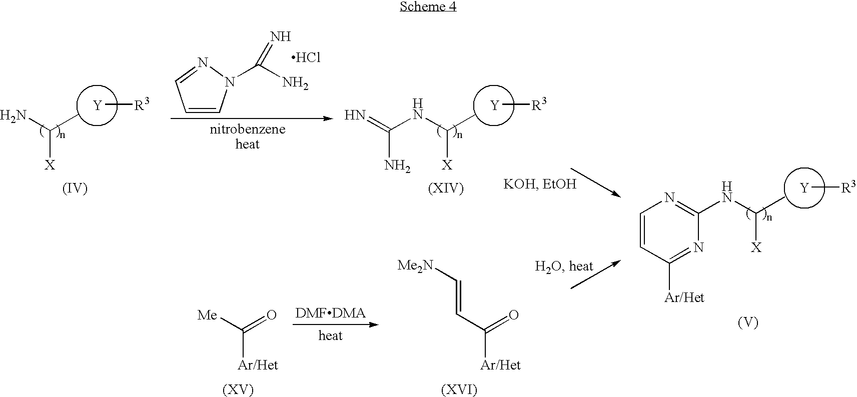

Aniline target molecules of structure (V) may also be prepared according to Scheme 4 (see, e.g., Bredereck, H. et al. Ber., Dtsch. Chem. Ges. 1964, 97, 3397, the disclosure of which is herein incorporated by reference). Anilines (IV) may be converted to the corresponding guanidines (XIV) using pyrazole-1-carboxamidine (see, e.g., Bernatowicz, M. S. et al. J. Org. Chem. 1992, 57, 2497, the disclosure of which is herein incorporated by reference). The guanidines may be combined with 3-dimethylamino-1-aryl/heteroaryl-propenones (XVI), prepared by heating methyl ketones (XV) with DMF DMA, in the presence of a base (e.g. KOH, NaOH, or Et₃N) or acid (e.g. HOAC in hot EtOH or MeOH) to give the desired 2-aminopyrimidines (V).

### EXAMPLES

The following general methods outline the synthesis of the heteroaryl/aryl pyrimidine analogs.

### General Experimental for the Preparation of 2-amino-4-aryl/heteroarylpyrimidines

### Procedure A

Step 1—Preparation of 2-chloro-4-aryl/heteroaryl-pyrimidine: To a −30° C. solution of a Ar/HetLi (10.66 mmol, 1.08 eq, generated via deprotonation of Li for Br exchange) in 20 ml of Et₂O was added portion-wise a suspension of 2-chloropyrimidine (9.84 mmol, 1 equiv.) in 20 ml Et₂O in 2 ml portions over 15 min. The resulting suspension was stirred for 30 min at −30° C. and at 0° C. for 60 min. The reaction was quenched with H₂O (0.27 ml, 1.5 equiv.) in THF (3 ml) and DDQ (2.95 g, 10.66 mmol, 1 equiv.) in THF (15 ml) was then added. The resulting suspension was stirred at 23° C. for 15 min, and then cooled to 0° C. Hexanes (10 ml) was added followed by and a 0° C. solution of NaOH (10 ml, 3N). The suspension was stirred for 5 min at 0° C., 100 ml of H₂O was added and the layers were separated. The organic layer was dried (Na₂SO₄) and concentrated in vacuo. Purification via SiO₂ gel column chromatography gave the title compound.

Step 2—Preparation of 2-amino-4-aryl/heteroarylpyrimidines: A 2-chloro-4-aryl/heteroaryl pyrimidine (0.26 mmol, 1 equiv.), amine (0.52 mmol, 2 equiv.), i-Pr₂NEt (1.3 mmol, 5 equiv.) and NMP (2 ml) solution were combined and heated at 90° C. for 12-18 h. Reaction progress was monitored using an analytical HP Agilent 1100 LC/MS according to the following method and parameters:

### HPLC: Analytical Method and Parameters:

### Instrument: HP Agilent 1100 LC/MS

### UV Detector: Agilent 1100 Diode Array Detector

### Mass Spectrometer Detector: Agilent MSD

Column: Waters Xterra MS C18 30 mm×2.1 mm i.d., 3.5 um
Flow Rate: 1.00 ml/min

### Run Time: 5.00 min

Gradient Elution: 0 min 90% water, 10% acetonitrile; 3 min 10% water, 90% acetonitrile

### Column Temperature: 50° C.

### UV Signals: 215 nm, 254 nm

### MS Parameters: Mass Range 100-1000, Fragmentor 140, Gain EMV 1.0.

After cooling to 23° C., all volatiles were removed in a Speed-Vac. This crude material was dissolved in 0.5 ml DMSO:1.5 ml MeCN, filtered through a 0.45 μm GMF, and purified on a Gilson HPLC, using a Phenomenex LUNA C₁₈ column: 60 mm×21.20 mm I.D., 5 μm particle size: with ACN/water (containing 0.2% TFA or Et₃N) gradient elution. The appropriate fractions were analyzed by LC/MS as described above. Combining pure fractions and evaporating the solvent in a Speed-Vac isolated the title compound.

### General Experimental for the Preparation of 2-anilino-4-aryl/heteroarylpyrimidine primary sulfonamides

### Procedure B

### Step 1

### Preparation of 3-dimethylamino-1-aryl/heteroaryl-propenone

A 0.1 M solution of a methyl ketone in DMF dimethylacetyl was heated at 130° C. for 12 h. After cooling to 23° C., all volatiles were evaporated. The remaining material was dissolved in a minimum of CH₂Cl₂ and passed through as short SPE SiO₂ gel cartridge eluting with additional CH₂Cl₂. The eluant was concentrated to a minimum volume and equal amount of hexanes was added. Cooling to 5° C. produces crystals of the title compound as a yellow or orange solid.

### Step 2

### Preparation of 2-amino-4-aryl/heteroarylpyrimidines

Amine (1 equiv.) was combined with 1.05 eq of 1H-pyrazole-1-carboxamidine hydrochloride and 2.05 equiv. of i-Pr₂NEt as a 0.1 M DMF solution and stirred at 23° C. for 6 h. Et₂O (10 ml) was added and the product guanidine precipitates/oils out. The solvent is removed and an additional 10 ml portion of Et₂O was added. The resulting precipitate was isolated by filtration and used directly in the next reaction.

To 1 equiv. of the guanidine and 1 equiv. of 3-dimethylamino-1-aryl/heteroaryl-propenone 1.25 equiv. of KOH, EtOH (equal volume to that of nitrobenzene) and H₂O ( 1/10^{th} the volume of EtOH) was added. This mixture was heated at 120° C. for 12 h, cooled to 23° C. and evaporated in a Speed-Vac. This crude material was dissolved in 0.5 ml DMSO: 1.5 ml MeCN, filtered through a 0.45 μm GMF, and purified on a Gilson HPLC, using a Phenomenex LUNA C₁₈ column: 60 mm×21.20 mm I.D., 5 um particle size: with ACN/water (containing 0.2% TFA or Et₃N) gradient elution. The appropriate fractions were analyzed by LC/MS as described above. Combining pure fractions and evaporating the solvent in a Speed-Vac isolated the title compound.

The following compounds were prepared according to the above procedures:

| | | LC Retention | | |
|---|---|---|---|---|
| Example | Name | Time (min)^{a} | ESMS Ion^{b} | Procedure |
| 1 | N-[3-(1H-Imidazol-1- | 1.08 | 281 (M + H)⁺ | B |
| | yl)propyl]-4-pyridin-4- | | | |
| | ylpyrimidin-2-amine | | | |
| 2 | N-[3-(1H-Imidazol-1- | 1.17 | 281 (M + H)⁺ | B |
| | yl)propyl]-4-pyridin-3- | | | |
| | ylpyrimidin-2-amine | | | |
| 3 | 2-Pyrimidinamine, N-[3-(1H- | 1.85 | 325 (M + H)⁺ | B |
| | imidazol-1-yl)propyl]-4-(3- | | | |
| | nitrophenyl)- | | | |
| 4 | 4-Pyrazin-2-yl-N-(2-pyridin- | 2.60 | 279 (M + H)⁺ | B |
| | 2-ylethyl)pyrimidin-2-amine | | | |
| 5 | 4-{3-[(4-Pyridin-2- | 2.23 | 295 (M − H)⁻ | B |
| | ylpyrimidin-2- | | | |
| | yl)amino]propyl}-2,4- | | | |
| | dihydro-3H-pyrazol-3-one | | | |
| 6 | N-[2-(1H-Imidazol-4- | 1.76 | 316 (M + H)⁺ | B |
| | yl)ethyl]-4-(2- | | | |
| | naphthyl)pyrimidin-2-amine | | | |
| 7 | N-[2-(1H-Imidazol-4- | 1.13 | 267 (M + H)⁺ | B |
| | yl)ethyl]-4-pyridin-3- | | | |
| | ylpyrimidin-2-amine | | | |
| 8 | (2S)-3-(1H-Imidazol-4-yl)-2- | 1.66 | 346 (M + H)⁺ | A |
| | {[4-(2-naphthyl)pyrimidin-2- | | | |
| | yl]amino}propan-1-ol | | | |
| 9 | N-[3-(3,5-Dimethyl-1H- | 2.6 | 358 (M + H)⁺ | B |
| | pyrazol-1-yl)propyl]-4-(2- | | | |
| | naphthyl)pyrimidin-2-amine | | | |
| 10 | N-[3-(3,5-Dimethyl-1H- | 1.68 | 309 (M + H)⁺ | B |
| | pyrazol-1-yl)propyl]-4- | | | |
| | pyridin-4-ylpyrimidin-2- | | | |
| | amine | | | |
| 11 | N-[2-(1H-Indol-3-yl)ethyl]-4- | 2.22 | 316 (M + H)⁺ | A |
| | pyridin-3-ylpyrimidin-2- | | | |
| | amine | | | |
| 12 | 2-Methyl-3-{2-[(4-pyridin-3- | 1.93 | 346 (M + H)⁺ | A |
| | ylpyrimidin-2-yl)amino]ethyl}- | | | |
| | 1H-indol-5-ol | | | |
| 13 | 4-Pyridin-3-yl-N-(2-pyridin-2- | 1.19 | 278 (M + H)⁺ | A |
| | ylethyl)pyrimidin-2-amine | | | |
| 14 | 4-Pyridin-3-yl-N-(2-pyridin-4- | 2.11 | 278 (M + H)⁺ | A |
| | ylethyl)pyrimidin-2-amine | | | |
| 15 | N-[2-(1H-Indol-3-yl)ethyl]-4- | 2.22 | 316 (M + H)⁺ | A |
| | pyridin-4-ylpyrimidin-2- | | | |
| | amine | | | |
| 16 | 4-Pyridin-4-yl-N-(2-pyridin-4- | 2.08 | 278 (M + H)⁺ | A |
| | ylethyl)pyrimidin-2-amine | | | |
| 17 | 4-(1-Benzothien-2-yl)-N-[2- | 2.57 | 322 (M + H)⁺ | A |
| | (1H-imidazol-4- | | | |
| | yl)ethyl]pyrimidin-2-amine | | | |
| 18 | 4-(1-Benzothien-2-yl)-N-(2- | 2.85 | 333 (M + H)⁺ | A |
| | pyridin-2-ylethyl)pyrimidin-2- | | | |
| | amine | | | |
| 19 | 4-(1-Naphthyl)-N-[2-(1H- | 1.80 | 366 (M + H)⁺ | A |
| | pyrrolo[2,3-c]pyridin-3- | | | |
| | yl)ethyl]pyrimidin-2-amine | | | |
| 20 | 4-(1-Naphthyl)-N-(2-pyridin- | 1.83 | 327 (M + H)⁺ | A |
| | 2-ylethyl)pyrimidin-2-amine | | | |
| 21 | 4-(5-Bromothien-2-yl)-N-(2- | 1.84 | 361 (M + H)⁺ | A |
| | pyridin-2-ylethyl)pyrimidin-2- | | | |
| | amine | | | |
| 22 | 4-(5-Bromothien-2-yl)-N-(2- | 1.79 | 361 (M + H)⁺ | A |
| | pyridin-4-ylethyl)pyrimidin-2- | | | |
| | amine | | | |

^{a}HPLC Conditions: Instrument - Agilent 1100; Column: Keystone Aquasil C18 (as above);
Mobile Phase A: 10 mM NH₄OAC in 95% water/5% CAN; Mobile Phase B: 10 mM
NH₄OAC in 5% water/95% CAN; Flow Rate: 0.800 ml/min; Column
Temperature: 40° C.; Injection Volume: 5 ul; UV: monitor 215, 230, 254, 280, and
300 nm; Purity is reported at 254 nm
unless otherwise noted.
Gradient Table:

| | |
|---|---|
| Time(min) | % B |
| 0.0 | 0 |
| 2.5 | 100 |
| 4.0 | 100 |
| 4.1 | 0 |
| 5.5 | 0 |

^{b}MS Conditions: Instrument: Agilent MSD; Ionization Mode: API-ES; Gas
Temperature: 350 C; Drying Gas: 11.0 L/min.; Nebulizer Pressure: 55psig; Polarity: 50%
positive, 50% negative; VCap: 3000V (positive), 2500V (negative); Fragmentor:
80 (positive), 120 (negative); Mass Range: 100-1000m/z; Threshold: 150;
Step size: 0.15; Gain: 1; Peak width: 0.15 min.

### General Experimental for the Preparation of N-substituted-3-(1H-imidazol-4-yl)-2-(4-naphthalen-2-yl-pyrimidin-2-ylamino)-propionamides

### Step 1

### Preparation of 3-(1H-imidazol-4-yl)-2-(4-naphthalen-2-yl-pyrimidin-2-ylamino)propionic acid

To 600 mg (2.4 mmol) of 2-chloro-4-naphthalen-2-yl-pyrimidine, prepared according to Procedure A Step 1, was added 410 mg (2.6 mmol) of DL-histidine, 24 ml of DMSO, followed by 384 mg (9.6 mmol) of 60% NaH in mineral oil. After heating at 80° C. for 12 h, all volatiles were removed in a Speed-Vac and the crude title compound was carried on directly to the next step.

### Step 2

### Preparation of N-substituted-3-(1H-imidazol-4-yl)-2-(4-naphthalen-2-yl-pyrimidin-2-ylamino)-propionamides

To 0.1 M DMF solution of 1 equiv. of 3-(1H-imidazol-4-yl)-2-(4-naphthalen-2-yl-pyrimidin-2-ylamino)-propionic acid was added 1.1 equiv. of amine, 2 equiv. of Et₃N followed by 1.5 equiv. of the BOP reagent. After stirring at 23° C. for 12 h, 2 ml of H₂O was added and the mixture was extracted with 2×5 ml of EtOAc. The combined organics were washed with 3×5 ml of H₂O and the solvent was evaporated to an oil. Purification by RP-HPLC as described in Procedure A, Step 2 gave the title compound.

The following compounds were prepared according to the above procedure:

| | | LC Retention | ESMS |
|---|---|---|---|
| Example | Name | Time (min)^{a} | Ion^{b} |
| 23 | N-Benzyl-N-[4-(2- | 2.09 | 449 |
| | naphthyl)pyrimidin-2- | | (M + H)⁺ |
| | yl]histidinamide | | |
| 24 | N-[2-(4-Methoxyphenyl)ethyl]- | — | 493 |
| | N-[4-(2-naphthyl)pyrimidin-2- | | (M + H)⁺ |
| | yl]histidinamide | | |

^{a}HPLC Conditions: Instrument - Agilent 1100; Column: Keystone
Aquasil C18 (as above); Mobile Phase A: 10 mM NH₄OAC in 95%
water/5% CAN; Mobile Phase B: 10 mM NH₄OAC in 5% water/95%
CAN: Flow Rate: 0.800 ml/min; Column Temperature: 40° C.; Injection
Volume: 5 ul; UV: monitor 215, 230, 254, 280, and 300 nm; Purity is
reported at 254 nm unless otherwise noted.
Gradient Table:

| | |
|---|---|
| Time(min) | % B |
| 0.0 | 0 |
| 2.5 | 100 |
| 4.0 | 100 |
| 4.1 | 0 |
| 5.5 | 0 |

^{b}MS Conditions: Instrument: Agilent MSD; Ionization Mode:
API-ES; Gas Temperature: 350° C.; Drying Gas: 11.0 L/min.;
Nebulizer Pressure: 55 psig; Polarity: 50% positive, 50% negative;
VCap: 3000 V (positive), 2500 V (negative); Fragmentor:
80 (positive), 120 (negative); Mass Range: 100-1000m/z;
Threshold: 150; Step size: 0.15; Gain: 1; Peak width: 0.15 min.

### General Experimental for the Preparation of 3-(1-substituted-1H-imidazol-4-yl)-2-(4-aryl/heteroaryl-pyrimidin-2-ylamino)-propan-1-ols

### Step 1

### Preparation of 3-(1H-Imidazol-4-yl)-2-(4-aryl/heteroaryl-pyrimidin-2-ylamino)-propan-1-ols

To a 0.1 M DMSO solution of 1 equiv. of a 2-chloro-4-aryl/heteroaryl-pyrimidine was added 1.2 equiv. of L-Histidinol and 4 equiv. of 60% NaH in mineral oil. After heating at 80° C. for 12 h, the reaction mixture was cooled to 23° C. All volatiles were removed using a Speed-Vac and crude title compound was carried on to the next step as a viscous oil.

### Step 2

### Preparation of 3-(1-substituted-1H-imidazol-4-yl)-2-(4-aryl/heteroaryl-pyrimidin-2-ylamino)-propan-1-ols

To a 0.1 M solution of 1 equiv. of 3-(1H-imidazol-4-yl)-2-(4-aryl/heteroaryl-pyrimidin-2-ylamino)-propan-1-ol was added 1 equiv of NaH (60% mineral oil dispersion). After stirring 23° C. for 30 min, 1.2 equiv. of the appropriate alkyl or benzyl halide was added. After stirring at 23° C. for 6-12 h, all volatiles were removed using a Speed-Vac and the resulting oil was purified by RP-HPLC as described in Procedure A, Step 2 to give the title compound.

The following compounds were prepared according to the above procedure:

| | | LC Retention | |
|---|---|---|---|
| Example | Name | Time (min)^{a} | ESMS Ion^{b} |
| 25 | Ethyl[4-((2S)-3-hydroxy-2-{[4-(2- | 2.07 | 432 (M + H)⁺ |
| | naphthyl)pyrimidin-2-yl]amino}propyl)-1H- | | |
| | imidazol-1-yl]acetate | | |
| 26 | (2S)-2-{[4-(2-Naphthyl)pyrimidin-2-yl]amino}-3-(1- | 3.05 | 384 (M + H)⁺ |
| | prop-2-yn-1-yl-1H-imidazol-4-yl)propan-1-ol | | |
| 27 | [4-((2S)-3-Hydroxy-2-{[4-(2-naphthyl)pyrimidin-2- | 1.74 | 385 (M + H)⁺ |
| | yl]amino}propyl)-1H-imidazol-1-yl]acetonitrile | | |
| 28 | 5-[4-((2S)-3-Hydroxy-2-{[4-(2-naphthyl)pyrimidin- | 1.74 | 427 (M + H)⁺ |
| | 2-yl]amino}propyl)-1H-imidazol-1- | | |
| | yl]pentanenitrile | | |
| 29 | 2-[4-((2S)-3-Hydroxy-2-{[4-(2-naphthyl)pyrimidin- | 1.64 | 403 (M + H)⁺ |
| | 2-yl]amino}propyl)-1H-imidazol-1-yl]acetamide | | |
| 30 | (2S)-3-[1-(3,5-Difluorobenzyl)-1H-imidazol-4-yl]- | 2.00 | 472 (M + H)⁺ |
| | 2-{[4-(2-naphthyl)pyrimidin-2-yl]amino}propan-1- | | |
| | ol | | |
| 31 | 1,1,1-Trifluoro-3-[4-((2S)-3-hydroxy-2-{[4-(2- | 2.14 | 456 (M + H)⁺ |
| | naphthyl)pyrimidin-2-yl]amino}propyl)-1H- | | |
| | imidazol-1-yl]acetone | | |
| 32 | 1-(3-{[4-((2S)-3-Hydroxy-2-{[4-(2- | 1.87 | 508 (M + H)⁺ |
| | naphthyl)pyrimidin-2-yl]amino}propyl)-1H- | | |
| | imidazol-1-yl]methyl}-4-methoxyphenyl)ethanone | | |
| 33 | (2S)-2-{[4-(1-Benzothien-2-yl)pyrimidin-2- | 3.06 | 390 (M + H)⁺ |
| | yl]amino}-3-(1-prop-2-yn-1-yl-1H-imidazol-4- | | |
| | yl)propan-1-ol | | |
| 34 | [4-((2S)-2-{[4-(1-Benzothien-2-yl)pyrimidin-2- | 1.70 | 391 (M + H)⁺ |
| | yl]amino}-3-hydroxypropyl)-1H-imidazol-1- | | |
| | yl]acetonitrile | | |
| 35 | 5-[4-((2S)-2-{[4-(1-Benzothien-2-yl)pyrimidin-2- | 1.73 | 433 (M + H)⁺ |
| | yl]amino}-3-hydroxypropyl)-1H-imidazol-1- | | |
| | yl]pentanenitrile | | |
| 36 | 2-[4-((2S)-2-{[4-(1-Benzothien-2-yl)pyrimidin-2- | 1.61 | 409 (M + H)⁺ |
| | yl]amino}-3-hydroxypropyl)-1H-imidazol-1- | | |
| | yl]acetamide | | |
| 37 | (2S)-2-{[4-(1-Benzothien-2-yl)pyrimidin-2- | 1.95 | 478 (M + H)⁺ |
| | yl]amino}-3-[1-(3,5-difluorobenzyl)-1H-imidazol- | | |
| | 4-yl]propan-1-ol | | |
| 38 | 1-(3-{[4-((2S)-2-{[4-(1-Benzothien-2-yl)pyrimidin- | 1.83 | 514 (M + H)⁺ |
| | 2-yl]amino}-3-hydroxypropyl)-1H-imidazol-1- | | |
| | yl]methyl}-4-methoxyphenyl)ethanone | | |
| 39 | 1-[4-((2S)-2-{[4-(1-Benzothien-2-yl)pyrimidin-2- | 1.78 | 424 (M + H)⁺ |
| | yl]amino}-3-hydroxypropyl)-1H-imidazol-1- | | |
| | yl]butan-2-ol | | |
| 40 | [4-((2S)-3-Hydroxy-2-{[4-(5-methyl-2- | 2.17 | 355 (M + H)⁺ |
| | thienyl)pyrimidin-2-yl]amino}propyl)-1H-imidazol- | | |
| | 1-yl]acetonitrile | | |
| 41 | 5-[4-((2S)-3-Hydroxy-2-{[4-(5-methyl-2- | 2.12 | 397 (M + H)⁺ |
| | thienyl)pyrimidin-2-yl]amino}propyl)-1H-imidazol- | | |
| | 1-yl]pentanenitrile | | |
| 42 | (2S)-3-[1-(4-Methylpent-3-en-1-yl)-1H-imidazol-4- | 1.74 | 398 (M + H)⁺ |
| | yl]-2-{[4-(5-methyl-2-thienyl)pyrimidin-2- | | |
| | yl]amino}propan-1-ol | | |
| 43 | (2S)-3-[1-(3-Cyclohexylpropyl)-1H-imidazol-4-yl]- | 1.98 | 440 (M + H)⁺ |
| | 2-{[4-(5-methyl-2-thienyl)pyrimidin-2- | | |
| | yl]amino}propan-1-ol | | |
| 44 | (2S)-3-[1-(3,5-Difluorobenzyl)-1H-imidazol-4-yl]- | 1.64 | 442 (M + H)⁺ |
| | 2-{[4-(5-methyl-2-thienyl)pyrimidin-2- | | |
| | yl]amino}propan-1-ol | | |
| 45 | 1-(3-{[4-((2S)-3-Hydroxy-2-{[4-(5-methyl-2- | 1.57 | 478 (M + H)⁺ |
| | thienyl)pyrimidin-2-yl]amino}propyl)-1H-imidazol- | | |
| | 1-yl]methyl}-4-methoxyphenyl)ethanone | | |
| 46 | (2S)-3-{1-[(2,6-Dichloropyridin-4-yl)methyl]-1H- | 1.66 | 475 (M + H)⁺ |
| | imidazol-4-yl}-2-{[4-(5-methyl-2-thienyl)pyrimidin- | | |
| | 2-yl]amino}propan-1-ol | | |
| 47 | 2-[4-((2S)-3-Hydroxy-2-{[4-(5-methyl-2- | 2.52 | 502 (M + H)⁺ |
| | thienyl)pyrimidin-2-yl]amino}propyl)-1H-imidazol- | | |
| | 1-yl]-1-[4-(trifluoromethyl)phenyl]ethanone | | |

^{a}HPLC Conditions: Instrument - Agilent 1100; Column: Keystone Aquasil C18
(as above); Mobile Phase A: 10 mM NH₄OAC in 95% water/5% CAN; Mobile
Phase B: 10 mM NH₄OAC in 5% water/95% CAN; Flow Rate: 0.800 ml/min;
Column Temperature: 40° C.; Injection Volume: 5 ul; UV: monitor 215, 230,
254, 280, and 300 nm; Purity is reported at 254 nm unless otherwise noted.
Gradient Table:

| | |
|---|---|
| Time(min) | % B |
| 0.0 | 0 |
| 2.5 | 100 |
| 4.0 | 100 |
| 4.1 | 0 |
| 5.5 | 0 |

^{b}MS Conditions: Instrument: Agilent MSD; Ionization Mode: API-ES; Gas
Temperature: 350° C.; Drying Gas: 11.0 L/min.; Nebulizer Pressure: 55psig;
Polarity: 50% positive, 50% negative; VCap: 3000V (positive), 2500V
(negative); Fragmentor: 80 (positive), 120 (negative); Mass Range: 100-1000m/z;
Threshold: 150; Step size: 0.15; Gain: 1; Peak width: 0.15 min.

### General Experimental for the Preparation of [2-substituted-1-(1H-imidazol-4-ylmethyl)-ethyl]-(4-naphthalen-2-yl-pyrimidin-2-yl)-amines

### Step 1

### Preparation of [1-alkoxy/aryloxymethyl-2-(1-trityl-1H-imidazol-4-yl)-ethyl]-trityl-amines

To a 0.1 M DMSO solution of 1 equiv. of 2-(trityl-amino)-3-(1-trityl-1H-imidazol-4-yl)-propan-1-ol (see, e.g., J. T. Kovalainen et al. J. Med. Chem. 1999, 42, 1193, the disclosure of which is herein incorporated by reference), was added 1.2 equiv. of alkyl, benzyl or heterobenzyl halides followed by 5.2 equiv. of 60% NaH in mineral oil. After stirring at 50° C. for 12 h, an additional portion of 1 equiv. of alkyl, benzyl or heterobenzyl halide was added. After stirring at 50° C. for an additional 12 h, the reaction mixture was cooled to 23° C. Water (25 ml) and ethyl acetate (25 ml) were added and the mixture was extracted. The aqueous portion was extracted with an additional 20 ml portion of ethyl acetate and the combined organics were washed with 4×25 ml of H₂O and evaporated to give the title compound as an off-white solid.

### Step 2

### Preparation of 2-(1H-Imidazol-4-yl)-1-alkoxy/aryloxymethyl-ethylamines dihydrochlorides

To a 0.1 M THF solution of 1 equiv. of a [1-alkoxy/aryloxymethyl-2-(1-trityl-1H-imidazol-4-yl)-ethyl]-trityl-amine was added 10 equiv. of 3 N HCl_{(aq)}, and the resulting mixture was heated to reflux. After 2 h reaction time, the reaction mixture was cooled to 23° C., all volatiles were removed in a Speed-Vac. Water (10 ml) was added, and the aqueous solution was extracted with 2×5 ml of CH₂Cl₂. The aqueous solution was evaporated to dryness in a Speed-Vac to give the title compound as an off-white solid.

### Step 3

### Preparation of 2-substituted-1-(1H-imidazol-4-ylmethyl)-ethyl]-(4-naphthalen-2-yl-pyrimidin-2-yl)-amines

The protocol of Procedure A, step 2 was used using 2-(1H-imidazol-4-yl)-1-alkoxy/aryloxymethyl-ethylamines dihydrochlorides as the amine.

The following compounds were prepared according to the above procedure:

| | | LC Retention | |
|---|---|---|---|
| Example | Name | Time (min)^{a} | ESMS Ion^{b} |
| 48 | N-[2-Ethoxy-1-(1H-imidazol-4-ylmethyl)ethyl]- | 2.05 | 375 (M + H)⁺ |
| | 4-(2-naphthyl)pyrimidin-2-amine | | |
| 49 | N-[2-(Cyclohexylmethoxy)-1-(1H-imidazol-4- | 2.48 | 442 (M + H)⁺ |
| | ylmethyl)ethyl]-4-(2-naphthyl)pyrimidin-2- | | |
| | amine | | |
| 50 | N-[2-[(5-tert-Butyl-1,2,4-oxadiazol-3- | 2.08 | 484 (M + H)⁺ |
| | yl)methoxy]-1-(1H-imidazol-4-ylmethyl)ethyl]- | | |
| | 4-(2-naphthyl)pyrimidin-2-amine | | |
| 51 | N-[2-{[2-Fluoro-3-(trifluoromethyl)benzyl]oxy}- | — | 522 (M + H)⁺ |
| | 1-(1H-imidazol-4-ylmethyl)ethyl]-4-(2- | | |
| | naphthyl)pyrimidin-2-amine | | |
| 52 | N-[2-(1H-Imidazol-4-yl)-1-({[5-(2- | 2.7 | 534 (M + H)⁺ |
| | methoxyphenyl)-1,2,4-oxadiazol-3- | | |
| | yl]methoxy}methyl)ethyl]-4-(2- | | |
| | naphthyl)pyrimidin-2-amine | | |
| 53 | 4-(1-Benzothien-2-yl)-N-{2-(1H-imidazol-4-yl)- | 2.02 | 390 (M + H)⁺ |
| | 1-[(prop-2-yn-1-yloxy)methyl]ethyl}pyrimidin- | | |
| | 2-amine | | |
| 54 | 4-(1-Benzothien-2-yl)-N-[2-[(5-tert-butyl-1,2,4- | 1.77 | 490 (M + H)⁺ |
| | oxadiazol-3-yl)methoxy]-1-(1H-imidazol-4- | | |
| | ylmethyl)ethyl]pyrimidin-2-amine | | |
| 55 | 2-{[2-{[4-(1-Benzothien-2-yl)pyrimidin-2- | — | 440 (M + H)⁺ |
| | yl]amino}-3-(1H-imidazol-4- | | |
| | yl)propoxy]methyl}benzonitrile | | |
| 56 | N-[2-[(5-tert-Butyl-1,2,4-oxadiazol-3- | 2.15 | 468 (M + H)⁺ |
| | yl)methoxy]-1-(1H-imidazol-4-ylmethyl)ethyl]- | | |
| | 4-(2-thienyl)pyrimidin-2-amine | | |

^{a}HPLC Conditions: Instrument - Agilent 1100; Column: Keystone Aquasil C18
(as above); Mobile Phase A: 10 mM NH₄OAC in 95% water/5% CAN; Mobile
Phase B: 10 mM NH₄OAC in 5% water/95% CAN: Flow Rate: 0.800 ml/min;
Column Temperature: 40° C.; Injection Volume: 5 ul; UV: monitor 215, 230,
254, 280, and 300 nm; Purity is reported at 254 nm unless otherwise noted.
Gradient Table:

| | |
|---|---|
| Time(min) | % B |
| 0.0 | 0 |
| 2.5 | 100 |
| 4.0 | 100 |
| 4.1 | 0 |
| 5.5 | 0 |

^{b}MS Conditions: Instrument: Agilent MSD; Ionization Mode: API-ES; Gas
Temperature: 350° C.; Drying Gas: 11.0 L/min.; Nebulizer Pressure: 55 psig;
Polarity: 50% positive, 50% negative; VCap: 3000 V (positive), 2500 V
(negative); Fragmentor: 80 (positive), 120 (negative); Mass Range: 100-1000m/z;
Threshold: 150; Step size: 0.15; Gain: 1; Peak width: 0.15 min.

### General Experimental for the Preparation of 2-amino-4-aryl/heteroarylpyrimidines

### Procedure C

### Step 1

### Preparation of 2-chloro-4-aryl/heteroaryl-pyrimidines

Sodium 4-(naphthalene-2-yl)pyrimidin-2-olate: 2-Acetyl naphthalene (15.0 g, 88.1 mmol) and DMF-dimethyl acetal (15.2 mL, 114.5 mmol) were combined and heated in an 85° C. bath overnight. The reaction was concentrated on a rotovap to a thick oil, which became a tan solid on standing under high vacuum. To the residue was added EtOH (anhydrous, 40 mL), urea (6.35 g, 105.7 mmol), and sodium ethoxide solution (21% weight solution in EtOH, 33 mL, 88.1 mmol) and the mixture was heated to gentle reflux overnight. The resulting mixture was cooled to room temperature, then filtered to collect a dark solid which was rinsed with EtOH. The solid was allowed to dry at room temperature for 1 h, then was suspended in H₂O and CH₂Cl₂ (1:1, ˜400 mL total). The resulting sticky material was collected by filtration and allowed to dry. An orange-pink powder (11.75 g, 54%) was obtained and carried on directly. LC/MS (Column; Xterra MS C18, 5μ, 50×2.1 mm. Mobile phase: 90/10-5/95 water (0.1% formic acid)/acetonitrile (0.1% formic acid), 2 min, hold 1.5 min, 0.8 mL/min., 210-400 nm) rt=1.10 mins., calculated mass=222, [M−H]⁻=221.

2-Chloro-4-(naphthalene-2-yl)pyrimidine: To thionyl chloride (56 mL, 0.77 mol) cooled in an ice bath was added the sodium salt of 4-(naphthalene-2-yl)pyrimidin-2(1H)-one (11.75 g, 48.1 mmol) in portions. To the mixture was added DMF (8 mL) and additional thionyl chloride (3 mL). The reaction was heated gradually in a 70° C. bath overnight. The solution was cooled to room temperature and concentrated in vacuo. Benzene (˜15 mL) was added and the solution was concentrated. This was repeated to give an orange solid. The solid was cooled in an ice bath and H₂O and K₂CO₃ were added to neutralize any acid. The material was extracted with CH₂Cl₂. The organic extracts were washed with brine, dried (Na₂SO₄), filtered, and concentrated to provide a brown solid (13.4 g). The solid was adsorbed onto silica gel (˜200 mL) and the silica was placed on a fritted funnel and was washed with 25% EtOAc/hexane (800 mL), then 50% EtOAc/hexane (400 mL). The 50% filtrate was concentrated to afford 4.2 g of a tan powder which was pure by ¹H NMR and LC/MS. The 25% filtrate was concentrated and the resulting solid was recrystallized from acetone to afford a first crop of 1.65 g and a second crop of 4 g of beige powder, which were pure by ¹H NMR and LC/MS. LC/MS (Column; Xterra MS C18, 5μ, 50×2.1 mm. Mobile phase: 90/10-5/95 water (0.1% formic acid)/acetonitrile (0.1% formic acid), 2 min, hold 1.5 min, 0.8 mL/min., 210-400 nm) rt=1.85 min., purity=96%, calculated mass=240, [M+H]⁺=241.

### Step 2

### Preparation of (2S)-3-(1H-indol-3-yl)-2-{[4-(2-thienyl)pyrimidin-2-yl]amino}propan-1-ol

To a solution of 2-chloro-4-(thien-2-yl)pyrimidine (51 mg, 0.26 mmol) in DMSO (0.85 mL) were added L-tryptophanol hydrochloride (235 mg, 1.04 mmol) and DIEA (217 μL, 1.24 mmol). The reaction was heated in a shaker block at 60° C. for 72 h. The crude reaction was purified by RP HPLC (YMC CombiPrep ProC18 50×20 mm I.D. column, S-5 μm, 12 nm. Flow rate 20 mL/min. Gradient: 10/90 Acetonitrile/Water to 100% acetonitrile over 10 minutes then hold for three minutes at 100% acetonitrile and ramp back to 10/90 acetonitrile/water over two minutes), followed by silica gel chromatography (2% MeOH/CH₂Cl₂ then 4% MeOH/CH₂Cl₂) to afford the title compound (27 mg, 30%) as a golden powder. HPLC (Column; Xterra RP18, 3.5μ, 150×4.6 mm. Mobile phase: 85/15-5/95 Ammonium formate buffer (pH=3.5)/ACN+MeOH (1:1) for 10 min, hold 4 mins, 1.2 mL/min., 210-370 nm) purity 100%, rt=9 min; LC/MS calculated mass=350, [M+H]⁺=351.

### General Experimental for the Preparation of 2-amino-4-aryl/heteroarylpyrimidines

### Procedure D

### Step 1

### Preparation of 2-methanesulfonyl-4-aryl/heteroaryl-pyrimidines

2-Methylsulfanyl-4-naphthalen-2-yl-pyrimidine: To a stirring solution of 2-chloro-4-naphthalen-2-yl-pyrimidine (2.84 g, 11.8 mmol) in anhydrous DMSO (40 mL) was added sodium thiomethoxide (2.48 g, 35.4 mmol) in anhydrous DMSO (20 mL) and the brown solution was stirred, at room temperature, under nitrogen for 3 h. The reaction was transferred to a separatory funnel and partitioned between ethyl acetate (100 mL) and water (100 mL). The aqueous layer was removed and the organic phase was washed five times with water (50 mL). The aqueous washings were combined and extracted five times with ethyl acetate (50 mL). The organic extracts were combined and washed with brine (100 mL), dried (MgSO₄), filtered and the solvent removed to give a tan solid (2.51 g, 84%) and carried on directly. LC/MS (Column; Xterra MS C18, 5μ, 50×2.1 mm. Mobile phase: 90/10-5/95 water (0.1% formic acid)/acetonitrile (0.1% formic acid), 2 min, hold 1.5 min, 0.8 mL/min., 210-400 nm), rt=1.97 mins., calculated mass=252, [M+H]⁺=253.

2-Methanesulfonyl-4-naphthalen-2-yl-pyrimidine: To a stirring solution of 2-methylsulfanyl-4-naphthalen-2-yl-pyrimidine (2.29 g, 9.08 mmol) in chloroform (100 mL) cooled in an ice bath, under nitrogen, was added 3-chloroperbenzoic acid (8.10 g, 46.9 mmol) and the solution allowed to warm to room temperature and stirred for 18 h. The reaction was transferred to a separatory funnel with chloroform (100 mL) and washed twice with sodium bisulfite (100 mL). The bisulfite washings were combined and extracted twice with chloroform (100 mL). The organic extracts were combined and washed twice with saturated sodium carbonate solution (150 mL), dried (MgSO₄), filtered and the solvent removed to give a light yellow solid which was adsorbed onto silica and purified by column chromatography (eluent: 50% ethyl acetate in hexane) to give a yellow solid (1.20 g, 46%). LC/MS (Column; Xterra MS C18, 5μ, 50×2.1 mm. Mobile phase: 90/10-5/95 water (0.1% formic acid)/acetonitrile (0.1% formic acid), 2 min, hold 1.5 min, 0.8 mL/min., 210-400 nm), rt=1.37 mins., calculated mass=284, [M+H]⁺=285.

### Step 2

### Preparation of Aminoalcohols

(2R)-2-Amino-3-(1H-indol-3-yl)-propan-1-ol: To a stirring mixture of D-tryptophan methyl ester (2.00 g, 7.85 mmol) in anhydrous THF (20 mL), under nitrogen, was added lithium borohydride [(2M in THF) 11.8 mL, 23.6 mmol]. The ester dissolved and the solution was stirred at room temperature, under nitrogen, for 72 h. Water was added to neutralize the lithium borohydride and the solvent was removed, in vacuo. The material was transferred to a separatory funnel with ethyl acetate (150 mL) and washed twice with a 1 M citric acid solution (100 mL). The aqueous washings were combined and extracted five times with ethyl acetate (75 mL). The organic extracts were combined and washed with brine (150 mL), dried (MgSO₄), filtered and the solvent removed, in vacuo, to give a white foam which was adsorbed onto silica and purified by column chromatography (eluent: 20% methanol in chloroform). This material remained impure and was purified using RP HPLC (YMC CombiPrep ProC18 50×20 mm I.D. column, S-5 μm, 12 nm. Flow rate 20 mL/min. Gradient: 10/90 Acetonitrile/Water to 100% acetonitrile over 10 minutes then hold for three minutes at 100% acetonitrile and ramp back to 10/90 acetonitrile/water over two minutes). The fractions were collected and the acetonitrile solvent was removed, in vacuo, and the remaining aqueous material lyophilized to give an amber colored oil (141 mg, 10%). HPLC (Column; Xterra RP18, 3.5μ, 150×4.6 mm. Mobile phase: 85/15-5/95 Ammonium formate buffer (pH=3.5)/ACN+MeOH (1:1) for 10 min, hold 4 mins, 1.2 mL/min., 210-370 nm), rt=3.5 mins., calculated mass=190, [M+H]⁺=191.

### Step 3

### Preparation of heteroaryl/aryl pyrimidine analogs

(2R)-3-(1H-indol-3-yl)-2-{[4-(2-naphthyl)pyrimidin-2-yl]amino}propan-1-ol: A solution of (2R)-2-amino-3-(1H-indol-3-yl)-propan-1-ol (67.2 mg, 0.35 mmol), 2-methanesulfonyl-4-naphthalen-2-yl-pyrimidine (83.7 mg, 0.29 mmol), and diisopropyl ethyl amine (107 μL, 0.62 mmol) in N-methylpyrrolidinone (5 mL) was heated in an 80° C. bath for 72 hours. The reaction was brought to room temperature and water (0.25 mL) was added. The mixture was purified by RP HPLC(YMC CombiPrep ProC18 50×20 mm I.D. column, S-5 μm, 12 nm. Flow rate 20 mL/min. Gradient: 10/90 Acetonitrile/Water (0.1% TFA in both solvents) to 100% acetonitrile over 10 minutes then hold for three minutes at 100% acetonitrile and ramp back to 10/90 acetonitrile/water over two minutes). The pure product fractions were concentrated and the residue was dissolved in ethyl acetate (150 mL) and washed with sodium carbonate solution (3×20 mL) and brine (20 mL). The organic layer was dried (Na₂SO₄), filtered, and concentrated to afford the title compound as a white solid (37.5 mg, 32%). HRMS: calcd for C₂₅H₂₂N₄O+H⁺, 395.18664; found (ESI, [M+H]⁺), 395.1868; HPLC (Column; Xterra RP18, 3.5μ, 150×4.6 mm. Mobile phase: 85/15-5/95 Ammonium formate buffer (pH=3.5)/ACN+MeOH (1:1) for 10 min, hold 4 min., 1.2 mL/min., 210-370 nm) rt=10.4 mins.

The following compounds were prepared by the previous methods:

| | | | ^{a}HPLC | |
|---|---|---|---|---|
| Example | | | R.t | Synthetic |
| no. | Compound Name | MS | (mins.) | Method |
| 57 | (S)-3-(1H-indol-3-yl)-2-(4- | 350.12 | 10.4 | D |
| | (thiophen-2-yl)pyrimidin-2- | | | |
| | ylamino)propan-1-ol | | | |
| 58 | (R)-3-(1H-indol-3-yl)-2-(4- | 394.2 | 9.0 | D |
| | (naphthalene-2-yl)pyrimidin- | | | |
| | 2-ylamino)propan-1-ol | | | |
| 59 | (2R)-2-{[4-(2- | 356.2 | 10.6 | D |
| | naphthyl)pyrimidin-2- | | | |
| | yl]amino}- | | | |
| | 3-phenylpropan-1-ol | | | |
| 60 | (2S)-3-(1H-indol-3-yl)-2-[(4- | 393.2 | 10.4 | D |
| | pyridin-4-ylpyrimidin-2- | | | |
| | yl)amino]propan-1-ol | | | |
| 61 | N-[2-(1H-indol-3-yl)ethyl]-4- | 365.2 | 11.4 | D |
| | (2-naphthyl)pyrimidin-2- | | | |
| | amine | | | |
| 62 | (2S)-3-(1H-indol-3-yl)-2-[(4- | 346.1 | 7.4 | C |
| | pyridin-4-ylpyrimidin-2- | | | |
| | yl)amino]propan-1-ol | | | |

^{a}HPLC Method: Column; Xterra RP18, 3.5μ, 150 × 4.6 mm. Mobile phase: 85/15-5/95 Ammonium formate buffer (pH = 3.5)/ACN + MeOH (1:1) for 10 min, hold 4 min, 1.2 mL/min., 210-370 nm.

### Biological Evaluation

### Functional Dkk1-LRP5-TCF-Luciferase Assay in U2OS Cells

Frozen U2OS-Dkk1-HTS Reporter cells generation: U2OS Human Bone derived cells (Osteosarcoma) were grown in McCoy's 5A Medium (Modified), with L-glutamine (GIBCO Cat No. 16600-082)+1% Pen-Strep+5% FBS) plated at 1×107 cells/T175 cm flask. The next day, the cells were co-transfected overnight with the following plasmids: (a) Test reporter (16xTCF-TK-FireFly-Luci), (b) Internal Control Reporter (TK-Renilla-Luci), (c) Wnt3a and (d) Dkk1. GIBCO's Lipofectamine 2000 and OptiMEM were used for the transfection. After a minimum of 4 hr of transfection at 37° C., the plasmid-transfected cells were trypsinized, counted, and suspended in freezing medium (95% FBS+5% DMSO). The reporter cells were frozen at 1×107/ml concentrations, aliquoted into 0.5 ml or 2.5 ml/tube and stored at −701C.

The following day, test compounds were added under HTS setup by Plate Track into 384 well plates (white, TC treated, Falcon plate) such that the final concentration of the compounds in 20 μL/well cell will be 5 μg/ml (final concentration of DMSO=0.25% and final compound concentration=20 μM). Vials of frozen reporter cells were thawed by warming the vial in a 37° C. water bath for 60-120 seconds with some shaking until the cells formed a suspension. The thawed cells were transferred into a cold 50 ml (or larger) tube and mixed well by gentle pipetting. The appropriate amount of cold Phenol Red Free RPMI medium-1640 (GIBCO, Cat # 11835-030) with L-glutamine was added, both with 5% FBS (GIBCO-BRL, Cat. # 16000-044), so that 20 μl of the final cell suspension will contain 5,000 cells. The cell dilution was done such that the final concentration of FBS was 5%. Diluted cells (20 μl) were added into each well in a 384 well plate. The plate was incubated at 37° C. under 5% CO₂ for 20 h. Bright-Glo substrate, 2.5 μl/well was added, and the Fire Fly Luciferase was measured using VLUX (60 second exposure) immediately after the substrate was added. Test compounds were dissolved in DMSO (100%) and added to specified wells. Raw luciferase signal data obtained as relative luminescence units (RLUs) for the test compounds were normalized to the signal of the mean of the sample reporter cell plate with DMSO.

Active compounds have TCF-luciferase ratios of 2.5 fold or greater over DMSO. All compounds show a signal increase of at least 10% compared to a signal with only DMSO added.

While particular embodiments of the present invention have been illustrated and described, it would be apparent to those skilled in the art that various other changes and modifications can be made without departing from the spirit and scope of the invention. It is therefore intended to cover in the appended claims all such changes and modifications that are within the scope of this invention.

Throughout this application, various publications are referenced. The disclosures of these publications in their entireties are hereby incorporated by reference into this application in order to more fully describe the state of the art as known to those skilled therein as of the date of the invention described and claimed herein.

What is claimed is:

1. A compound of the Formula (1): 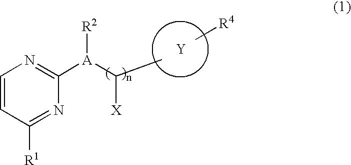
or pharmaceutically acceptable salts, hydrates, or solvates thereof,
wherein
R¹ is aryl, or heteroaryl, both optionally substituted by 1-7 R⁴ groups;
A is CH, N, O, S, SO or SO₂;
n is 1-4;
R² is H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H;
Y is imidazolyl, triazolyl, pyrazolyl, pyrrolyl, pyridinyl, pyrimidinyl, pyridizinyl, pyrazinyl, phenyl, indolyl, pyrolepyridinyl, all optionally substituted by 1-6 independently selected R⁴ groups; and
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

2. The compound of claim 1, wherein R¹ is optionally substituted aryl.

3. The compound of claim 1, wherein R¹ is optionally substituted heteroaryl.

4. The compound of claim 1, wherein A is nitrogen.

5. The compound of claim 1, wherein the compound of Formula (1) has the Formula (1A): 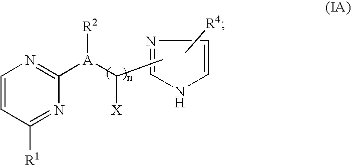
wherein the imidazolyl ring is optionally substituted with 1-3 R⁴ groups.

6. The compound of claim 5, wherein the compound is selected from the group consisting of
N-[3-(1H-imidazol-1-yl)propyl]-4-(4-pyridinyl)-2-pyrimidinamine;
N-[3-(1H-imidazol-1-yl)propyl]-4-(3-pyridinyl)-2-pyrimidinamine;
N-[3-(1H-imidazol-1-yl)propyl]-4-(3-nitrophenyl)-2-p+C10yrimidinamine;
N-[2-(1H-imidazol-4-yl)ethyl]-4-(2-naphthyl)pyrimidin-2-amine;
N-[2-(1H-imidazol-4-yl)ethyl]-4-pyridin-3-ylpyrimidin-2-amine;
(2S)-3-(1H-imidazol-4-yl)-2-{[4-(2-naphthyl)pyrimidin-2-yl]amino}propan-1-ol;
4-(1-benzothien-2-yl)-N-[2-(1H-imidazol-4-yl)ethyl]pyrimidin-2-amine;
N-benzyl-N-[4-(2-naphthyl)pyrimidin-2-yl]histidinamide;
N-[2-(4-methoxyphenyl)ethyl]-N-[4-(2-naphthyl)pyrimidin-2-yl]histidinamide;
ethyl[4-((2S)-3-hydroxy-2-{[4-(2-naphthyl)pyrimidin-2-yl]amino}propyl)-1H-imidazol-1-yl]acetate;
(2S)-2-{[4-(2-naphthyl)pyrimidin-2-yl]amino}-3-(1-prop-2-yn-1-yl-1H-imidazol-4-yl)propan-1-ol;
[4-((2S)-3-hydroxy-2-{[4-(2-naphthyl)pyrimidin-2-yl]amino}propyl)-1H-imidazol-1-yl]acetonitrile;
5-[4-((2S)-3-hydroxy-2-{[4-(2-naphthyl)pyrimidin-2-yl]amino}propyl)-1H-imidazol-1-yl]pentanenitrile;
2-[4-((2S)-3-hydroxy-2-{[4-(2-naphthyl)pyrimidin-2-yl]amino}propyl)-1H-imidazol-1-yl]acetamide;
(2S)-3-[1-(3,5-difluorobenzyl)-1H-imidazol-4-yl]-2-{[4-(2-naphthyl)pyrimidin-2-yl]amino}propan-1-ol;
1,1,1-trifluoro-3-[4-((2S)-3-hydroxy-2-{[4-(2-naphthyl)pyrimidin-2-yl]amino}propyl)-1H-imidazol-1-yl]acetone;
1-(3-{[4-((2S)-3-hydroxy-2-{[4-(2-naphthyl)pyrimidin-2-yl]amino}propyl)-1H-imidazol-1-yl]methyl}-4-methoxyphenyl)ethanone;
(2S)-2-{[4-(1-benzothien-2-yl)pyrimidin-2-yl]amino}-3-(1-prop-2-yn-1-yl-1H-imidazol-4-yl)propan-1-ol;
[4-((2S)-2-{[4-(1-benzothien-2-yl)pyrimidin-2-yl]amino}-3-hydroxypropyl)-1H-imidazol-1-yl]acetonitrile;
5-[4-((2S)-2-{[4-(1-benzothien-2-yl)pyrimidin-2-yl]amino}-3-hydroxypropyl)-1H-imidazol-1-yl]pentanenitrile;
2-[4-((2S)-2-{[4-(1-benzothien-2-yl)pyrimidin-2-yl]amino}-3-hydroxypropyl)-1H-imidazol-1-yl]acetamide;
(2S)-2-{[4-(1-benzothien-2-yl)pyrimidin-2-yl]amino}-3-[1-(3,5-difluorobenzyl)-1H-imidazol-4-yl]propan-1-ol;
1-(3-{[4-((2S)-2-{[4-(1-benzothien-2-yl)pyrimidin-2-yl]amino}-3-hydroxypropyl)-1H-imidazol-1-yl]methyl}-4-methoxyphenyl)ethanone;
1-[4-((2S)-2-{[4-(1-benzothien-2-yl)pyrimidin-2-yl]amino}-3-hydroxypropyl)-1H-imidazol-1-yl]butan-2-ol;
[4-((2S)-3-hydroxy-2-{[4-(5-methyl-2-thienyl)pyrimidin-2-yl]amino}propyl)-1H-imidazol-1-yl]acetonitrile;
5-[4-((2S)-3-hydroxy-2-{[4-(5-methyl-2-thienyl)pyrimidin-2-yl]amino}propyl)-1H-imidazol-1-yl]pentanenitrile;
(2S)-3-[1-(4-methylpent-3-en-1-yl)-1H-imidazol-4-yl]-2-{[4-(5-methyl-2-thienyl)pyrimidin-2-yl]amino}propan-1-ol;
(2S)-3-[1-(3-cyclohexylpropyl)-1H-imidazol-4-yl]-2-{[4-(5-methyl-2-thienyl)pyrimidin-2-yl]amino}propan-1-ol;
(2S)-3-[1-(3,5-difluorobenzyl)-1H-imidazol-4-yl]-2-{[4-(5-methyl-2-thienyl)pyrimidin-2-yl]amino}propan-1-ol;
1-(3-{[4-((2S)-3-hydroxy-2-{[4-(5-methyl-2-thienyl)pyrimidin-2-yl]amino}propyl)-1H-imidazol-1-yl]methyl}-4-methoxyphenyl)ethanone;
(2S)-3-{1-[(2,6-dichloropyridin-4-yl)methyl]-1H-imidazol-4-yl}-2-{[4-(5-methyl-2-thienyl)pyrimidin-2-yl]amino}propan-1-ol;
2-[4-((2S)-3-hydroxy-2-{[4-(5-methyl-2-thienyl)pyrimidin-2-yl]amino}propyl)-1H-imidazol-1-yl]-1-[4-(trifluoromethyl)phenyl]ethanone;
N-[2-ethoxy-1-(1H-imidazol-4-ylmethyl)ethyl]-4-(2-naphthyl)pyrimidin-2-amine;
N-[2-(cyclohexylmethoxy)-1-(1H-imidazol-4-ylmethyl)ethyl]-4-(2-naphthyl)pyrimidin-2-amine;
N-[2-[(5-tert-butyl-1,2,4-oxadiazol-3-yl)methoxy]-1-(1H-imidazol-4-ylmethyl)ethyl]-4-(2-naphthyl)pyrimidin-2-amine;
N-[2-{[2-fluoro-3-(trifluoromethyl)benzyl]oxy}-1-(1H-imidazol-4-ylmethyl)ethyl]-4-(2-naphthyl)pyrimidin-2-amine;
N-[2-(1H-imidazol-4-yl)-1-({[5-(2-methoxyphenyl)-1,2,4-oxadiazol-3-yl]methoxy}methyl)ethyl]-4-(2-naphthyl)pyrimidin-2-amine;
4-(1-benzothien-2-yl)-N-{2-(1H-imidazol-4-yl)-1-[(prop-2-yn-1-yloxy)methyl]ethyl}pyrimidin-2-amine;
4-(1-benzothien-2-yl)-N-[2-[(5-tert-butyl-1,2,4-oxadiazol-3-yl)methoxy]-1-(1H-imidazol-4-ylmethyl)ethyl]pyrimidin-2-amine;
2-{[2-{[4-(1-benzothien-2-yl)pyrimidin-2-yl]amino}-3-(1H-imidazol-4-yl)propoxy]methyl}benzonitrile; and
N-[2-[(5-tert-butyl-1,2,4-oxadiazol-3-yl)methoxy]-1-(1H-imidazol-4-ylmethyl)ethyl]-4-(2-thienyl)pyrimidin-2-amine.

7. The compound of claim 1, wherein the compound of Formula (1) has the Formula (1B): 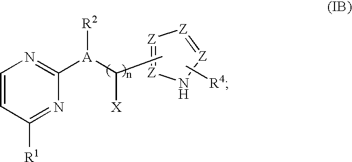
wherein each Z is independently N or CH, whereby two of the Z groups are N, thereby forming a triazolyl ring;
wherein the triazolyl ring is optionally substituted with 1-2 independently selected R⁴ groups.

8. The compound of claim 1, wherein the compound of Formula (1) has the Formula (1C): 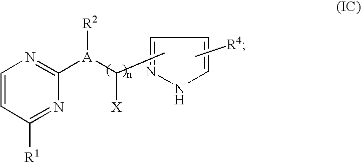
wherein the pyrazolyl ring is optionally substituted with 1-3 independently selected R⁴ groups.

9. The compound of claim 8, wherein the compound is selected from the group consisting of
N-[3-(3,5-dimethyl-1H-pyrazol-1-yl)propyl]-4-(2-naphthyl)pyrimidin-2-amine;
N-[3-(3,5-dimethyl-1H-pyrazol-1-yl)propyl]-4-pyridin-4-ylpyrimidin-2-amine; and
4-{3-[(4-pyridin-2-ylpyrimidin-2-yl)amino]propyl}-2,4-dihydro-3H-pyrazol-3-one.

10. The compound of claim 1, wherein the compound of Formula (1) has the Formula (1D): 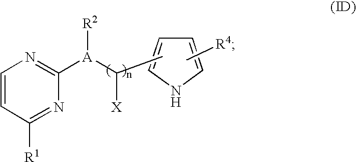
wherein the pyrrolyl ring is optionally substituted with 1-3 independently selected R⁴ groups.

11. The compound of claim 1, wherein the compound of Formula (1) has the Formula (1E): 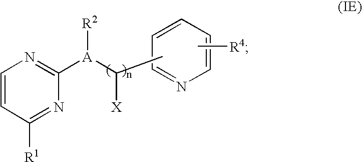
wherein the pyridinyl ring is optionally substituted with 1-4 independently selected R⁴ groups.

12. The compound of claim 11, wherein the compound is selected from the group consisting of
4-pyrazin-2-yl-N-(2-pyridin-2-ylethyl)pyrimidin-2-amine;
4-pyridin-3-yl-N-(2-pyridin-2-ylethyl)pyrimidin-2-amine;
4-pyridin-3-yl-N-(2-pyridin-4-ylethyl)pyrimidin-2-amine;
4-pyridin-4-yl-N-(2-pyridin-4-ylethyl)pyrimidin-2-amine;
4-(1-benzothien-2-yl)-N-(2-pyridin-2-ylethyl)pyrimidin-2-amine;
4-(1-naphthyl)-N-(2-pyridin-2-ylethyl)pyrimidin-2-amine;
4-(5-bromothien-2-yl)-N-(2-pyridin-2-ylethyl)pyrimidin-2-amine;
4-(5-bromothien-2-yl)-N-(2-pyridin-4-ylethyl)pyrimidin-2-amine;
4-(naphthalen-2-yl)-N-(pyridin-3-ylmethyl)pyrimidin-2-amine;
N-(2-(pyridin-3-yl)ethyl)-4-(pyridin-4-yl)pyrimidin-2-amine; and
N-(2-(pyridin-3-yl)ethyl)-4-(thiophen-2-yl)pyrimidin-2-amine.

13. The compound of claim 1, wherein the compound of Formula (1) has the Formula (1F): 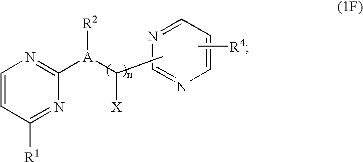
wherein the pyrimidinyl ring is optionally substituted with 1-3 independently selected R⁴ groups.

14. The compound of claim 1, wherein the compound of Formula (1) has the Formula (1G): 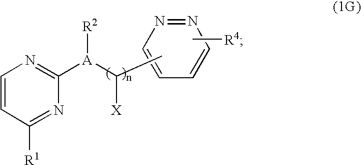
wherein the pyridizinyl ring is optionally substituted with 1-3 independently selected R⁴ groups.

15. The compound of claim 1, wherein the compound of Formula (1) has the Formula (1H): 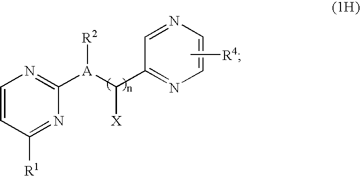
wherein the pyrazinyl ring is optionally substituted with 1-3 independently selected R⁴ groups.

16. The compound of claim 1, wherein the compound of Formula (1) has the Formula (1I): 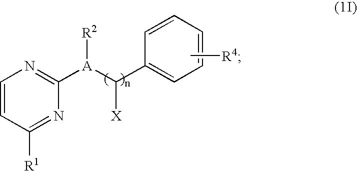
wherein the phenyl ring is optionally substituted with 1-5 independently selected R⁴ groups.

17. The compound of claim 16, wherein the compound is (2R)-2-{[4-(2-naphthyl)pyrimidin-2-yl]amino}-3-phenylpropan-1-ol.

18. The compound of claim 1, wherein the compound of Formula (1) has the Formula (1J): 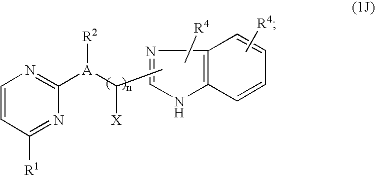
wherein the indolyl ring is optionally substituted with 1-6 independently selected R⁴ groups.

19. The compound of claim 18, wherein the compound is selected from the group consisting of
N-[2-(1H-indol-3-yl)ethyl]-4-pyridin-3-ylpyrimidin-2-amine;
2-methyl-3-{2-[(4-pyridin-3-ylpyrimidin-2-yl)amino]ethyl}-1H-indol-5-ol;
N-[2-(1H-indol-3-yl)ethyl]-4-pyridin-4-ylpyrimidin-2-amine;
(2S)-3-(1H-indol-3-yl)-2-{[4-(2-thienyl)pyrimidin-2-yl]amino}propan-1-ol;
(2R)-3-(1H-indol-3-yl)-2-{[4-(2-naphthyl)pyrimidin-2-yl]amino}propan-1-ol;
(2S)-3-(1H-indol-3-yl)-2-{[4-(2-naphthyl)pyrimidin-2-yl]amino}propan-1-ol;
N-[2-(1H-indol-3-yl)ethyl]-4-(2-naphthyl)pyrimidin-2-amine; and
(2S)-3-(1H-indol-3-yl)-2-[(4-pyridin-4-ylpyrimidin-2-yl)amino]propan-1-ol.

19. The compound of claim 1, wherein the compound of Formula (1) has the Formula (1K): 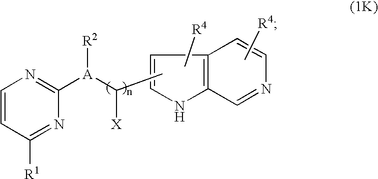
wherein the pyrrolpyridinyl ring is optionally substituted with 1-4 independently selected R⁴ groups.

20. The compound of claim 19, wherein the compound is 4-(1-naphthyl)-N-[2-(1H-pyrrolo[2,3-c]pyridin-3-yl)ethyl]pyrimidin-2-amine.

21. A pharmaceutical composition comprising the compound or pharmaceutically acceptable salt of the compound of claim 1 and a pharmaceutically acceptable carrier.

22. The pharmaceutical composition of claim 21, wherein the pharmaceutically acceptable carrier is suitable for oral administration and the pharmaceutical composition comprises an oral dosage form.

23. A method of treating a canonical Wnt-β-catenin cellular messaging system related disorder, comprising administering to a mammal in need thereof a compound or a pharmaceutically acceptable salt of a compound of claim 1 in an amount effect to treat a canonical Wnt-β-catenin cellular messaging system related disorder.

24. The method of claim 23, wherein the canonical Wnt-β-catenin cellular messaging system related disorder is selected from the group consisting of bone disorders, cancer, and Alzheimer's disease.

25. The method of claim 24, wherein the canonical Wnt-β-catenin cellular messaging system related disorder is cancer.

26. The method of claim 25, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

27. A canonical Wnt-β-catenin cellular messaging system agonist of claim 1.

28. A method of synthesizing a compound of Formula (1), comprising:
reacting a compound of the Formula (2): 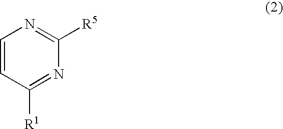
wherein
R¹ is aryl or heteroaryl, both optionally substituted by 1-7 R⁴ groups;
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl; and,
R⁵ is halogen;
with a compound of Formula (3): 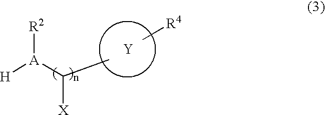
wherein
A is CH, N, O, S, SO or SO₂;
n is 1-4;
R²H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H, provided that when A is SO₂, R² is null;
each X is independently H, CON(R³)₂, or CH₂OR³;
each R³ is independently H, C₁-C₆ alkyl, C₂-C₆ alkenyl, C₂-C₆ alkynyl, aryl, arylalkyl, heteroaryl, or heteroarylalkyl, wherein all are optionally substituted by 1-5 R⁴ groups except H;
Y is imidazolyl, triazolyl, pyrazolyl, pyrrolyl, pyridinyl, pyrimidinyl, pyridizinyl, pyrazinyl, phenyl, indolyl, pyrolepyridinyl, all optionally substituted by 1-6 R⁴ groups; and
each R⁴ is independently H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl;
under conditions effective to substitute R⁵ with the compound of Formula 3 thereby providing a compound having the Formula 1: 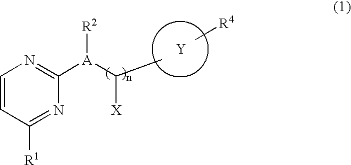
or pharmaceutically acceptable salts, hydrates, or solvates thereof.
